SEQUENCE LISTING (119 sequences)

SEQ ID NO: 1 - protein, 99 aa, synthetic construct
  REGION 1..99: note = Ankyrin repeat domain
NDTKGATPLHLAAVEGHLEIVEVLLKHGADVNAMDNQGQTPLHLAAQWGHLEIVEVLLKYGADVNAADIAGYTPLHLAAWFGHLEIVEVLLKYGVDVNA

SEQ ID NO: 2 - protein, 99 aa, synthetic construct
  REGION 1..99: note = Ankyrin repeat domain
SDWHGATPLHLAAEQGHLEIVEVLLKHGADVNATDESGNTPLHLAAVFGHLEIVEVLLKNGADVNASDHFGLTPLHLAAWFGHLEIVEVLLKHGADVNA

SEQ ID NO: 3 - protein, 47 aa, synthetic construct
  REGION 1..47: note = Ankyrin repeat domain
TDKAGFTPLHLAAYVGHLEIVEVLLKNGADVNAHDHQGDTSLHLAAT

SEQ ID NO: 4 - protein, 99 aa, synthetic construct
  REGION 1..99: note = Ankyrin repeat domain
TDEEGLTPLHLAALIGHLEIVEVLLKYGADVNAADMDGTTPLHLAAQEGHLEIVEVLLKYGADVNADDFWGHTPPHLAAWFGHLEIVEVLLKHGADVNA

SEQ ID NO: 5 - protein, 99 aa, synthetic construct
  REGION 1..99: note = Ankyrin repeat domain
TDEEGLTPLHLAALIGHLEIVEVLLKYGADVNAADMDGTTPLHLAAQEGHLEIVEVLLKYGADVNADDFWGHTPLHLAAWFGHLEIVEVLLKHGADVDA

SEQ ID NO: 6 - protein, 99 aa, synthetic construct
  REGION 1..99: note = Ankyrin repeat domain
TDEEGLTPLHLAALIGHLEIVEVLLKYGADVNAADMDGTTPLHLAAQEGHLEIVEVLLKYGADVNADDFWGHTPLHLAAWFGHLEIVEVLLKHGADVNA

SEQ ID NO: 7 - protein, 99 aa, synthetic construct
  REGION 1..99: note = Ankyrin repeat domain
TDEEGLTPLHLAALIGHLEIVEVLLKYGADVNAADMDGTTPLHLAAQEGHLEIVEVLLKYGADVNADDFWGHTPLHLAAWFGHLEIAEVLLKHGTDVNA

SEQ ID NO: 8 - protein, 99 aa, synthetic construct
  REGION 1..99: note = Ankyrin repeat domain
WDKEGVTPLHLAAVHGHLEIVEVLLKHGADVNAQDDVGMTPLHLAAHWGHLEIVEVLLKYGADVNAQDYAGMTPLHLAAFQGHLEIVEVLLKHGADVNA

SEQ ID NO: 9 - protein, 99 aa, synthetic construct
  REGION 1..99: note = Ankyrin repeat domain
TDNEGQTPLHLAAMWGHLEIVEVQLKHGADVNASDTFGSTPLHLAARFGHLEIVEVLLKYGADVNAADTEGFTPLHLAAIDGHLEIVEVLLKHGADVNA

SEQ ID NO: 10 - protein, 99 aa, synthetic construct
  REGION 1..99: note = Ankyrin repeat domain
TDITGATPLHLAAMWGHLEIVEVLLKYGADVNAQDYEGYTTLHLAAMWGHLEIVEVLLKYGADVNAHDVAGHTPLHLATHFGHLEIVEVLLKHGADVNA

SEQ ID NO: 11 - protein, 99 aa, synthetic construct; identical to SEQ ID NO: 8
  REGION 1..99: note = Ankyrin repeat domain

SEQ ID NO: 12 - protein, 99 aa, synthetic construct
  REGION 1..99: note = Ankyrin repeat domain
YDHMGETPLHLAAMEGHLEIVEVLLKHGADVNADDWDGQTPLHLAAHWGHLEIVEVLLKYGADVKEQDVVGYTPLHLAAWIGHLEIVEVLLKNGADVNA

SEQ ID NO: 13 - protein, 99 aa, synthetic construct
  REGION 1..99: note = Ankyrin repeat domain
TDEEGLTPLHLAALDGHLEIVEVLLKYGADVNADDMHGPTPLHLAAYAGHLEIVEVLLKYGADVNAYDCGGHTPLHLAARLGHLEIVEVLLKYGADVNA

SEQ ID NO: 14 - protein, 99 aa, synthetic construct
  REGION 1..99: note = Ankyrin repeat domain
TDEEGLTPLHLAVLIGHLEIVDVLLKYGADVNAADMDGTTPLHLAAQEGHLEIVEVLLKYGADVNADDFWGHTPLHLAAWFGHLEIVEVLLKHGADVNA

SEQ ID NO: 15 - protein, 99 aa, synthetic construct
  REGION 1..99: note = Ankyrin repeat domain
TDEEGLTPLHLAALIGHLEIVEVLLKYGADVNAVDMDGTTPLHLAAQEGHLEIVEVLLKYGADVNADDFWGHTPLHLAAWFGHLEIVEVLLKHGADVNA

SEQ ID NO: 16 - protein, 99 aa, synthetic construct
  REGION 1..99: note = Ankyrin repeat domain
TDEEGLTPLHLAALIGHPEIVEVLLKYGADVNAADMDGTTPLHLAAQEGHLEIVEVLLKYCADVNADDFWGHTPLHLAAWFGHLEIVEVLLKYGADVNA

SEQ ID NO: 17 - protein, 99 aa, synthetic construct
  REGION 1..99: note = Ankyrin repeat domain
TDEEGLTPLHLAALIGHLEIVEVLLKYGADVNAADMDGTTPLHLAAQEGHLEIVEVLLKYGADVNADDFWGHTPLHLAAWFGHLEIVEVLLKNGADVNA

SEQ ID NO: 18 - protein, 99 aa, synthetic construct
  REGION 1..99: note = Ankyrin repeat domain
TDEEGLTPLHLAALIGHLEIVEVLLKYGADVNAADMDGTTPLHLAAQEGHLEIVEVLLKYGADVNADDFWGHTPLHLAAWFGHLEIVDVLLANVADVNA

SEQ ID NO: 19 - protein, 99 aa, synthetic construct
  REGION 1..99: note = Ankyrin repeat domain
TDEEGLTPLHLAALIGHLEIVEVLLKYGADVNAADMDGTTPLHLAVQEGHLEIVEVLLKYGADVNADDFWGHTPLHLAAWFGHLEIVEVLLKYGADVNA

SEQ ID NO: 20 - protein, 99 aa, synthetic construct; identical to SEQ ID NO: 17
  REGION 1..99: note = Ankyrin repeat domain

SEQ ID NO: 21 - protein, 99 aa, synthetic construct
  REGION 1..99: note = Ankyrin repeat domain
TDEEGLTPLHLAALIGHLEIVEVLLKYGTDVNAADMDGTTPLHLAAQEGHLEIVEVLLKYGADVNADDFWGHTPLHLAAWFGHLEIVEVLLKHGADVNA

SEQ ID NO: 22 - protein, 99 aa, synthetic construct
  REGION 1..99: note = Ankyrin repeat domain
TDEEGLTPLHLAALIGHLEIVEVLLKYGADVNAADMDGTTPLHLAAQEGHQEIVEVLLKYGADVNADDFWGHTPLHLAAWFGHLEIVEVLLKHGADVNA

SEQ ID NO: 23 - protein, 99 aa, synthetic construct; identical to SEQ ID NO: 6
  REGION 1..99: note = Ankyrin repeat domain

SEQ ID NO: 24 - protein, 99 aa, synthetic construct
  REGION 1..99: note = Ankyrin repeat domain
TDEEGLTPLHLAALIGHLEIVEVLLKYDADVNAADMDGTTPLHLAAQEGHLEIVEVLLKYGADVNADDFWGHTPLHLAPWFGHLEIVEVLLNHGADVNA

SEQ ID NO: 25 - protein, 99 aa, synthetic construct; identical to SEQ ID NO: 6
  REGION 1..99: note = Ankyrin repeat domain

SEQ ID NO: 26 - protein, 99 aa, synthetic construct; identical to SEQ ID NO: 6
  REGION 1..99: note = Ankyrin repeat domain

SEQ ID NO: 27 - protein, 66 aa, synthetic construct
  REGION 1..66: note = Ankyrin repeat domain
EDNFGTTPLHLAAQFGHLEIVEVLLKNGVDVNAHDYWGRTPLYLAAEYGHLEIVEVLLKYGADVNA

SEQ ID NO: 28 - protein, 33 aa, synthetic construct
  REGION 1..33: note = Ankyrin repeat unit 1
TDEEGLTPLHLAALIGHLEIVEVLLKYGADVNA

SEQ ID NO: 29 - protein, 33 aa, synthetic construct
  REGION 1..33: note = Ankyrin repeat unit 2
ADMDGTTPLHLAAQEGHLEIVEVLLKYGADVNA

SEQ ID NO: 30 - protein, 33 aa, synthetic construct
  REGION 1..33: note = Ankyrin repeat unit 3
DDFWGHTPLHLAAWFGHLEIVEVLLKHGADVNA

SEQ ID NO: 31 - protein, 33 aa, synthetic construct
  REGION 1..33: note = Ankyrin repeat sequence motif
  VARIANT 1: note = X1 can be A, R, N, D, B, E, Q, Z, H, I, L, K, M, F, S, T, W, Y
  VARIANT 3: note = X2 can be A, R, N, D, B, E, Q, Z, H, I, L, K, M, F, S, T, W, Y
  VARIANT 4: note = X3 can be A, R, N, D, B, E, Q, Z, H, I, L, K, M, F, S, T, W, Y
  VARIANT 6: note = X4 can be A, R, N, D, B, E, Q, Z, H, I, L, K, M, F, S, T, W, Y
  VARIANT 14: note = X5 can be A, R, N, D, B, E, Q, Z, H, I, L, K, M, F, S, T, W, Y
  VARIANT 15: note = X6 can be A, R, N, D, B, E, Q, Z, H, I, L, K, M, F, S, T, W, Y
  VARIANT 27: note = X7 can be H, N, Y
XDXXGXTPLHLAAXXGHLEIVEVLLKXGADVNA

SEQ ID NO: 32 - protein, 30 aa, synthetic construct
  REGION 1..30: note = N-cap reference sequence
DLGKKLLEAARAGQDDEVRILMANGADVNA

SEQ ID NO: 33 - protein, 28 aa, synthetic construct
  REGION 1..28: note = C-cap reference sequence
QDKFGKTPFDLAIDNGNEDIAEVLQKAA

SEQ ID NO: 34 - protein, 30 aa, synthetic construct; identical to SEQ ID NO: 32
  REGION 1..30: note = N-cap repeat 53.H3

SEQ ID NO: 35 - protein, 30 aa, synthetic construct; identical to SEQ ID NO: 32
  REGION 1..30: note = N-cap repeat 53.2.F11

SEQ ID NO: 36 - protein, 30 aa, synthetic construct; identical to SEQ ID NO: 32
  REGION 1..30: note = N-cap repeat 53.C8

SEQ ID NO: 37 - protein, 30 aa, synthetic construct
  REGION 1..30: note = N-cap repeat 53.D8 N-cap
DLGKKLLEATRAGQDDEVRILMANGADVNA

SEQ ID NO: 38 - protein, 30 aa, synthetic construct; identical to SEQ ID NO: 37
  REGION 1..30: note = N-cap repeat 53.2.A8

SEQ ID NO: 39 - protein, 30 aa, synthetic construct
  REGION 1..30: note = N-cap repeat 53.2.D6
DLGKKLLDAASAGQDDEVRILMANGADVNA

SEQ ID NO: 40 - protein, 30 aa, synthetic construct; identical to SEQ ID NO: 32
  REGION 1..30: note = N-cap repeat 53.2.G2

SEQ ID NO: 41 - protein, 30 aa, synthetic construct
  REGION 1..30: note = N-cap repeat 53.A10
DLGKKLLEAARAGQDDEVRILMSNGADVNA

SEQ ID NO: 42 - protein, 30 aa, synthetic construct
  REGION 1..30: note = N-cap repeat 53.C1
DLGKKLLEAARVGQDDEVRILMANGADVNA

SEQ ID NO: 43 - protein, 30 aa, synthetic construct; identical to SEQ ID NO: 32
  REGION 1..30: note = N-cap repeat 53.D11

SEQ ID NO: 44 - protein, 30 aa, synthetic construct
  REGION 1..30: note = N-cap repeat 53.G1
DLGKKLLEATRAGQDDEVRILMSNGADVNA

SEQ ID NO: 45 - protein, 30 aa, synthetic construct
  REGION 1..30: note = N-cap repeat 53.2.C8
DLGKKLLEAARAGQDDAVRILMANGADANA

SEQ ID NO: 46 - protein, 30 aa, synthetic construct
  REGION 1..30: note = N-cap repeat 53.A8
DLGKKLLEATRAGQDDEVRILMANGANVNA

SEQ ID NO: 47 - protein, 30 aa, synthetic construct; identical to SEQ ID NO: 37
  REGION 1..30: note = N-cap repeat 53.G7

SEQ ID NO: 48 - protein, 30 aa, synthetic construct; identical to SEQ ID NO: 37
  REGION 1..30: note = N-cap repeat 53.H9

SEQ ID NO: 49 - protein, 30 aa, synthetic construct
  REGION 1..30: note = N-cap repeat 53.B8
DLGKKLLEATRAGQDDEVRILMTNGTDVNA

SEQ ID NO: 50 - protein, 30 aa, synthetic construct; identical to SEQ ID NO: 37
  REGION 1..30: note = N-cap repeat 53.B10

SEQ ID NO: 51 - protein, 30 aa, synthetic construct
  REGION 1..30: note = N-cap repeat 53.2.H4
DLGKKLLEATRAGQDDEVRILMANGADVNT

SEQ ID NO: 52 - protein, 30 aa, synthetic construct; identical to SEQ ID NO: 37
  REGION 1..30: note = N-cap repeat 53.2.H9

SEQ ID NO: 53 - protein, 30 aa, synthetic construct; identical to SEQ ID NO: 32
  REGION 1..30: note = N-cap repeat 53.D7

SEQ ID NO: 54 - protein, 30 aa, synthetic construct; identical to SEQ ID NO: 32
  REGION 1..30: note = N-cap repeat 53.E11

SEQ ID NO: 55 - protein, 30 aa, synthetic construct; identical to SEQ ID NO: 37
  REGION 1..30: note = N-cap repeat 53.2.B6

SEQ ID NO: 56 - protein, 30 aa, synthetic construct; identical to SEQ ID NO: 51
  REGION 1..30: note = N-cap repeat 53.2.E10

SEQ ID NO: 57 - protein, 30 aa, synthetic construct; identical to SEQ ID NO: 37
  REGION 1..30: note = N-cap repeat 53.2.H5

SEQ ID NO: 58 - protein, 30 aa, synthetic construct; identical to SEQ ID NO: 37
  REGION 1..30: note = N-cap repeat 53.F11; 53.D10

SEQ ID NO: 59 - protein, 30 aa, synthetic construct; identical to SEQ ID NO: 37
  REGION 1..30: note = N-cap repeat 53.A11

SEQ ID NO: 60 - protein, 30 aa, synthetic construct
  REGION 1..30: note = N-cap repeat 52.H6
DLGKKLLEAARAGQDDEVRILIANGADVNA

SEQ ID NO: 61 - protein, 28 aa, synthetic construct
  REGION 1..28: note = C-cap repeat 53.H3
QDKFGKTPFDLAIDNGNEDIAEVLQKAA

SEQ ID NO: 62 - protein, 28 aa, synthetic construct
  REGION 1..28: note = C-cap repeat 53.2.F11
QDKFGKTPFDLAIDNGNEDIAEVLQKAA

SEQ ID NO: 63 - protein, 28 aa, synthetic construct
  REGION 1..28: note = C-cap repeat 53.D8
QDKFGKTPFDLAIDNGNEDIAEVLQKAA

SEQ ID NO: 64 - protein, 28 aa, synthetic construct
  REGION 1..28: note = C-cap repeat 53.2.A8
QDKFGKTPFDLAIDNGNEDIAEVLQKAA

SEQ ID NO: 65 - protein, 28 aa, synthetic construct
  REGION 1..28: note = C-cap repeat 53.2.D6
QDKFGKTPFDLAIDNGNEDIAEVLQKAA

SEQ ID NO: 66 - protein, 28 aa, synthetic construct
  REGION 1..28: note = C-cap repeat 53.2.G2
QDKFGKTPFDLAIDNGNEDIAEVLQKAA

SEQ ID NO: 67 - protein, 28 aa, synthetic construct
  REGION 1..28: note = C-cap repeat 53.A10
QDKFGKTPFDLAIDNGNEDIAQVLQKAA

SEQ ID NO: 68 - protein, 28 aa, synthetic construct
  REGION 1..28: note = C-cap repeat 53.C1
QDKFGKTPFDLAIDNGNEDIAEVLQKAA

SEQ ID NO: 69 - protein, 28 aa, synthetic construct
  REGION 1..28: note = C-cap repeat 53.D11
QDKFGKTPFDLAIDNGNEDIAEVLQKAA

SEQ ID NO: 70 - protein, 28 aa, synthetic construct
  REGION 1..28: note = C-cap repeat 53.G1
QDKFGKTPFDLAIDNGNEDIAEVLQKAA

SEQ ID NO: 71 - protein, 28 aa, synthetic construct
  REGION 1..28: note = C-cap repeat 53.2.C8
QDKFGKTPFDLAIDNGNEDIAEVLQKAA

SEQ ID NO: 72 - protein, 28 aa, synthetic construct
  REGION 1..28: note = C-cap repeat 53.A8
QDKFGKTPFDLAIDNGNEDIAEVLQKAA

SEQ ID NO: 73 - protein, 28 aa, synthetic construct
  REGION 1..28: note = C-cap repeat 53.G7
QDKFGKTPFDLAIDNGNEDIAEVLQKAA

SEQ ID NO: 74 - protein, 28 aa, synthetic construct
  REGION 1..28: note = C-cap repeat 53.H9
QDKFGKTPFDLAIDNGNEDIAQVLQKAA

SEQ ID NO: 75 - protein, 28 aa, synthetic construct
  REGION 1..28: note = C-cap repeat 53.B8
QDKFGKTPFDLAIDNGNEDIAEVLQKAA

SEQ ID NO: 76 - protein, 28 aa, synthetic construct
  REGION 1..28: note = C-cap repeat 53.B10
QDKFGKTPFDLAIDNGNEDIAEVLQKAA

SEQ ID NO: 77 - protein, 28 aa, synthetic construct
  REGION 1..28: note = C-cap repeat 53.2.H4
NSAQSKTPFDLAIDNGNEDIAEVLQKAA

SEQ ID NO: 78 - protein, 28 aa, synthetic construct
  REGION 1..28: note = C-cap repeat 53.2.H9
QDKFGKTPFDLAIDNGNEDIAEALQKAA

SEQ ID NO: 79 - protein, 28 aa, synthetic construct
  REGION 1..28: note = C-cap repeat 53.D7
QDKFGKTPFDLAIDNGNEDIAEVLQKAA

SEQ ID NO: 80 - protein, 28 aa, synthetic construct
  REGION 1..28: note = C-cap repeat 53.E11
QDKFGKTPFDLAIDNGNEDIAEVLQKAA

SEQ ID NO: 81 - protein, 28 aa, synthetic construct
  REGION 1..28: note = C-cap repeat 53.2.B6
QDKFGKIPFDLAIDNGNEDIAEVLQKAA

SEQ ID NO: 82 - protein, 28 aa, synthetic construct
  REGION 1..28: note = C-cap repeat 53.2.E10
QDKFGKTPFDLAIDNGNEDIAEVLQKAA

SEQ ID NO: 83 - protein, 28 aa, synthetic construct
  REGION 1..28: note = C-cap repeat 53.2.H5
QDKFGKTPFDLAIDNGNEDLAEVLQKAA

SEQ ID NO: 84 - protein, 28 aa, synthetic construct
  REGION 1..28: note = C-cap repeat 53.F11; 53.D10
QDKFGKTPFDLAIDNGNEDIAQVLQKAA

SEQ ID NO: 85 - protein, 28 aa, synthetic construct
  REGION 1..28: note = C-cap repeat 53.A11
QDKFGKTPFDLAIDNGNEDIAEVLQKAA

SEQ ID NO: 86 - protein, 30 aa, synthetic construct
  REGION 1..30: note = C-cap repeat 52.H6
QDKFGKTPFDLAIDNGNEDIAEMLQKAAAA

SEQ ID NO: 87 - protein, 317 aa, Homo sapiens
MTMETQMSQNVCPRNLWLLQPLTVLLLLASADSQAAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSHLDPTFSIPQANHSHSGDYHCTGNIGYTLFSSKPVTITVQVPSMGSSSPMGIIVAVVIATAVAAIVAAVVALIYCRKKRISANSTDPVKAAQFEPPGRQMIAIRKRQLEETNNDYETADGGYMTLNPRAPTDDDKNIYLTLPPNDHVNSNN

SEQ ID NO: 88 - protein, 310 aa, Homo sapiens
MGILSFLPVLATESDWADCKSPQPWGHMLLWTAVLFLAPVAGTPAAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGIIVAVVTGIAVAAIVAAVVALIYCRKKRISALPGYPECREMGETLPEKPANPTNPDEADKVGAENTITYSLLMHPDALEEPDDQNRI

SEQ ID NO: 89 - protein, 323 aa, Homo sapiens
MGILSFLPVLATESDWADCKSPQPWGHMLLWTAVLFLAPVAGTPAAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGIIVAVVTGIAVAAIVAAVVALIYCRKKRISANSTDPVKAAQFEPPGRQMIAIRKRQPEETNNDYETADGGYMTLNPRAPTDDDKNIYLTLPPNDHVNSNN

SEQ ID NO: 90 - protein, 157 aa, synthetic construct
  REGION 1..157: note = 53.H3 full darpin
DLGKKLLEAARAGQDDEVRILMANGADVNANDTKGATPLHLAAVEGHLEIVEVLLKHGADVNAMDNQGQTPLHLAAQWGHLEIVEVLLKYGADVNAADIAGYTPLHLAAWFGHLEIVEVLLKYGVDVNAQDKFGKTPFDLAIDNGNEDIAEVLQKAA

SEQ ID NO: 91 - protein, 157 aa, synthetic construct
  REGION 1..157: note = 53.2.F11 full darpin
DLGKKLLEAARAGQDDEVRILMANGADVNASDWHGATPLHLAAEQGHLEIVEVLLKHGADVNATDESGNTPLHLAAVFGHLEIVEVLLKNGADVNASDHFGLTPLHLAAWFGHLEIVEVLLKHGADVNAQDKFGKTPFDLAIDNGNEDIAEVLQKAA

SEQ ID NO: 92 - protein, 77 aa, synthetic construct
  REGION 1..77: note = 53.C8 full darpin
DLGKKLLEAARAGQDDEVRILMANGADVNATDKAGFTPLHLAAYVGHLEIVEVLLKNGADVNAHDHQGDTSLHLAAT

SEQ ID NO: 93 - protein, 157 aa, synthetic construct
  REGION 1..157: note = 53.D8 full darpin
DLGKKLLEATRAGQDDEVRILMANGADVNATDEEGLTPLHLAALIGHLEIVEVLLKYGADVNAADMDGTTPLHLAAQEGHLEIVEVLLKYGADVNADDFWGHTPPHLAAWFGHLEIVEVLLKHGADVNAQDKFGKTPFDLAIDNGNEDIAEVLQKAA

SEQ ID NO: 94 - protein, 157 aa, synthetic construct
  REGION 1..157: note = 53.2.A8 full darpin
DLGKKLLEATRAGQDDEVRILMANGADVNATDEEGLTPLHLAALIGHLEIVEVLLKYGADVNAADMDGTTPLHLAAQEGHLEIVEVLLKYGADVNADDFWGHTPLHLAAWFGHLEIVEVLLKHGADVDAQDKFGKTPFDLAIDNGNEDIAEVLQKAA

SEQ ID NO: 95 - protein, 157 aa, synthetic construct
  REGION 1..157: note = 53.2.D6 full darpin
DLGKKLLDAASAGQDDEVRILMANGADVNATDEEGLTPLHLAALIGHLEIVEVLLKYGADVNAADMDGTTPLHLAAQEGHLEIVEVLLKYGADVNADDFWGHTPLHLAAWFGHLEIVEVLLKHGADVNAQDKFGKTPFDLAIDNGNEDIAEVLQKAA

SEQ ID NO: 96 - protein, 157 aa, synthetic construct
  REGION 1..157: note = 53.2.G2 full darpin
DLGKKLLEAARAGQDDEVRILMANGADVNATDEEGLTPLHLAALIGHLEIVEVLLKYGADVNAADMDGTTPLHLAAQEGHLEIVEVLLKYGADVNADDFWGHTPLHLAAWFGHLEIAEVLLKHGTDVNAQDKFGKTPFDLAIDNGNEDIAEVLQKAA

SEQ ID NO: 97 - protein, 157 aa, synthetic construct
  REGION 1..157: note = 53.A10 full darpin
DLGKKLLEAARAGQDDEVRILMSNGADVNAWDKEGVTPLHLAAVHGHLEIVEVLLKHGADVNAQDDVGMTPLHLAAHWGHLEIVEVLLKYGADVNAQDYAGMTPLHLAAFQGHLEIVEVLLKHGADVNAQDKFGKTPFDLAIDNGNEDIAQVLQKAA

SEQ ID NO: 98 - protein, 157 aa, synthetic construct
  REGION 1..157: note = 53.C1 full darpin
DLGKKLLEAARVGQDDEVRILMANGADVNATDNEGQTPLHLAAMWGHLEIVEVQLKHGADVNASDTFGSTPLHLAARFGHLEIVEVLLKYGADVNAADTEGFTPLHLAAIDGHLEIVEVLLKHGADVNAQDKFGKTPFDLAIDNGNEDIAEVLQKAA

SEQ ID NO: 99 - protein, 157 aa, synthetic construct
  REGION 1..157: note = 53.D11 full darpin
DLGKKLLEAARAGQDDEVRILMANGADVNATDITGATPLHLAAMWGHLEIVEVLLKYGADVNAQDYEGYTTLHLAAMWGHLEIVEVLLKYGADVNAHDVAGHTPLHLATHFGHLEIVEVLLKHGADVNAQDKFGKTPFDLAIDNGNEDIAEVLQKAA

SEQ ID NO: 100 - protein, 157 aa, synthetic construct
  REGION 1..157: note = 53.G1 full darpin
DLGKKLLEATRAGQDDEVRILMSNGADVNAWDKEGVTPLHLAAVHGHLEIVEVLLKHGADVNAQDDVGMTPLHLAAHWGHLEIVEVLLKYGADVNAQDYAGMTPLHLAAFQGHLEIVEVLLKHGADVNAQDKFGKTPFDLAIDNGNEDIAEVLQKAA

SEQ ID NO: 101 - protein, 157 aa, synthetic construct
  REGION 1..157: note = 53.2.C8 full darpin
DLGKKLLEAARAGQDDAVRILMANGADANAYDHMGETPLHLAAMEGHLEIVEVLLKHGADVNADDWDGQTPLHLAAHWGHLEIVEVLLKYGADVKEQDVVGYTPLHLAAWIGHLEIVEVLLKNGADVNAQDKFGKTPFDLAIDNGNEDIAEVLQKAA

SEQ ID NO: 102 - protein, 157 aa, synthetic construct
  REGION 1..157: note = 53.A8 full darpin
DLGKKLLEATRAGQDDEVRILMANGANVNATDEEGLTPLHLAALDGHLEIVEVLLKYGADVNADDMHGPTPLHLAAYAGHLEIVEVLLKYGADVNAYDCGGHTPLHLAARLGHLEIVEVLLKYGADVNAQDKFGKTPFDLAIDNGNEDIAEVLQKAA

SEQ ID NO: 103 - protein, 157 aa, synthetic construct
  REGION 1..157: note = 53.G7 full darpin
DLGKKLLEATRAGQDDEVRILMANGADVNATDEEGLTPLHLAVLIGHLEIVDVLLKYGADVNAADMDGTTPLHLAAQEGHLEIVEVLLKYGADVNADDFWGHTPLHLAAWFGHLEIVEVLLKHGADVNAQDKFGKTPFDLAIDNGNEDIAEVLQKAA

SEQ ID NO: 104 - protein, 157 aa, synthetic construct
  REGION 1..157: note = 53.H9 full darpin
DLGKKLLEATRAGQDDEVRILMANGADVNATDEEGLTPLHLAALIGHLEIVEVLLKYGADVNAVDMDGTTPLHLAAQEGHLEIVEVLLKYGADVNADDFWGHTPLHLAAWFGHLEIVEVLLKHGADVNAQDKFGKTPFDLAIDNGNEDIAQVLQKAA

SEQ ID NO: 105 - protein, 157 aa, synthetic construct
  REGION 1..157: note = 53.B8 full darpin
DLGKKLLEATRAGQDDEVRILMTNGTDVNATDEEGLTPLHLAALIGHPEIVEVLLKYGADVNAADMDGTTPLHLAAQEGHLEIVEVLLKYCADVNADDFWGHTPLHLAAWFGHLEIVEVLLKYGADVNAQDKFGKTPFDLAIDNGNEDIAEVLQKAA

SEQ ID NO: 106 - protein, 157 aa, synthetic construct
  REGION 1..157: note = 53.B10 full darpin
DLGKKLLEATRAGQDDEVRILMANGADVNATDEEGLTPLHLAALIGHLEIVEVLLKYGADVNAADMDGTTPLHLAAQEGHLEIVEVLLKYGADVNADDFWGHTPLHLAAWFGHLEIVEVLLKNGADVNAQDKFGKTPFDLAIDNGNEDIAEVLQKAA

SEQ ID NO: 107 - protein, 157 aa, synthetic construct
  REGION 1..157: note = 53.2.H4 full darpin
DLGKKLLEATRAGQDDEVRILMANGADVNTTDEEGLTPLHLAALIGHLEIVEVLLKYGADVNAADMDGTTPLHLAAQEGHLEIVEVLLKYGADVNADDFWGHTPLHLAAWFGHLEIVDVLLANVADVNANSAQSKTPFDLAIDNGNEDIAEVLQKAA

SEQ ID NO: 108 - protein, 157 aa, synthetic construct
  REGION 1..157: note = 53.2.H9 full darpin
DLGKKLLEATRAGQDDEVRILMANGADVNATDEEGLTPLHLAALIGHLEIVEVLLKYGADVNAADMDGTTPLHLAVQEGHLEIVEVLLKYGADVNADDFWGHTPLHLAAWFGHLEIVEVLLKYGADVNAQDKFGKTPFDLAIDNGNEDIAEALQKAA

SEQ ID NO: 109 - protein, 157 aa, synthetic construct
  REGION 1..157: note = 53.D7 full darpin
DLGKKLLEAARAGQDDEVRILMANGADVNATDEEGLTPLHLAALIGHLEIVEVLLKYGADVNAADMDGTTPLHLAAQEGHLEIVEVLLKYGADVNADDFWGHTPLHLAAWFGHLEIVEVLLKNGADVNAQDKFGKTPFDLAIDNGNEDIAEVLQKAA

SEQ ID NO: 110 - protein, 157 aa, synthetic construct
  REGION 1..157: note = 53.E11 full darpin
DLGKKLLEAARAGQDDEVRILMANGADVNATDEEGLTPLHLAALIGHLEIVEVLLKYGTDVNAADMDGTTPLHLAAQEGHLEIVEVLLKYGADVNADDFWGHTPLHLAAWFGHLEIVEVLLKHGADVNAQDKFGKTPFDLAIDNGNEDIAEVLQKAA

SEQ ID NO: 111 - protein, 157 aa, synthetic construct
  REGION 1..157: note = 53.2.B6 full darpin
DLGKKLLEATRAGQDDEVRILMANGADVNATDEEGLTPLHLAALIGHLEIVEVLLKYGADVNAADMDGTTPLHLAAQEGHQEIVEVLLKYGADVNADDFWGHTPLHLAAWFGHLEIVEVLLKHGADVNAQDKFGKIPFDLAIDNGNEDIAEVLQKAA

SEQ ID NO: 112 - protein, 157 aa, synthetic construct
  REGION 1..157: note = 53.2.E10 full darpin
DLGKKLLEATRAGQDDEVRILMANGADVNTTDEEGLTPLHLAALIGHLEIVEVLLKYGADVNAADMDGTTPLHLAAQEGHLEIVEVLLKYGADVNADDFWGHTPLHLAAWFGHLEIVEVLLKHGADVNAQDKFGKTPFDLAIDNGNEDIAEVLQKAA

SEQ ID NO: 113 - protein, 157 aa, synthetic construct
  REGION 1..157: note = 53.2.H5 full darpin
DLGKKLLEATRAGQDDEVRILMANGADVNATDEEGLTPLHLAALIGHLEIVEVLLKYDADVNAADMDGTTPLHLAAQEGHLEIVEVLLKYGADVNADDFWGHTPLHLAPWFGHLEIVEVLLNHGADVNAQDKFGKTPFDLAIDNGNEDLAEVLQKAA

SEQ ID NO: 114 - protein, 157 aa, synthetic construct
  REGION 1..157: note = 53.F11; 53.D10 full darpin
DLGKKLLEATRAGQDDEVRILMANGADVNATDEEGLTPLHLAALIGHLEIVEVLLKYGADVNAADMDGTTPLHLAAQEGHLEIVEVLLKYGADVNADDFWGHTPLHLAAWFGHLEIVEVLLKHGADVNAQDKFGKTPFDLAIDNGNEDIAQVLQKAA

SEQ ID NO: 115 - protein, 157 aa, synthetic construct
  REGION 1..157: note = 53.A11...full darpin
DLGKKLLEATRAGQDDEVRILMANGADVNATDEEGLTPLHLAALIGHLEIVEVLLKYGADVNAADMDGTTPLHLAAQEGHLEIVEVLLKYGADVNADDFWGHTPLHLAAWFGHLEIVEVLLKHGADVNAQDKFGKTPFDLAIDNGNEDIAEVLQKAA

SEQ ID NO: 116 - protein, 126 aa, synthetic construct
  REGION 1..126: note = 52.H6 full darpin
DLGKKLLEAARAGQDDEVRILIANGADVNAEDNFGTTPLHLAAQFGHLEIVEVLLKNGVDVNAHDYWGRTPLYLAAEYGHLEIVEVLLKYGADVNAQDKFGKTPFDLAIDNGNEDIAEMLQKAAAA

SEQ ID NO: 117 - protein, 184 aa, Homo sapiens
QAAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSHLDPTFSIPQANHSHSGDYHCTGNIGYTLFSSKPVTITVQVPSMGSSSPMG

SEQ ID NO: 118 - protein, 175 aa, Homo sapiens
TPAAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAP

SEQ ID NO: 119 - protein, 181 aa, Homo sapiens
TPAAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMG

(19) United States
(12) Patent Application Publication
(10) Pub. No.: US 2025/0101071 A1
(43) Pub. Date: Mar. 27, 2025

(54) BINDING PROTEINS SPECIFIC FOR CD32A

(71) Applicant: PAUL-EHRLICH-INSTITUT, BUNDESINSTITUT FÜR IMPFSTOFFE UND BIOMEDIZINISCHE ARZNEIMITTEL, Langen (DE)

(72) Inventors: Vanessa RIECHERT, Langen (DE); Jessiaca HARTMANN, Bad Soden (DE); Christian BUCHHOLZ, Frankfurt am Main (DE); Klaus CICHUTEK, Langen (DE)

(21) Appl. No.: 18/728,187

(22) PCT Filed: Jan. 13, 2023
(86) PCT No.: PCT/EP2023/050760

§ 371 Date: Jul. 11, 2024

(30) Foreign Application Priority Data
Jan. 14, 2022 (EP) 22151678.4

(51) Int. Cl. C07K 14/47; A61K 38/00; G01N 33/68

(52) U.S. Cl. CPC C07K 14/47; G01N 33/68; A61K 38/00; C07K 2318/20; G01N 2333/70535

(57) ABSTRACT

The present invention relates to binding proteins comprising ankyrin repeats, in particular such that specifically bind CD32a or specifically target CD32a positive cells. Nucleic acids encoding such binding proteins, nucleic acids, vectors comprising said nucleic acids, particles comprising said binding protein, a host cell comprising said nucleic acid, a pharmaceutical composition are disclosed and any of these for use in medicine and/or several gene therapies.

The present invention relates to binding proteins with binding specificity for CD32a, as well as nucleic acids encoding such binding proteins, vectors comprising said nucleic acids, said binding proteins for use in medicine and/or gene therapy; and a pharmaceutical composition comprising said binding proteins, nucleic acids, or vectors. Particularly, the present invention relates to said binding proteins, which specifically binds CD32a and not CD32b.

### BACKGROUND OF THE INVENTION

CD32 (also known as FcγRII or FCGR2) is a surface receptor glycoprotein that can be found on a variety of immune cells, for example on platelets, neutrophils, macrophages, and dendritic cells. In humans, there are three major CD32 subtypes: CD32a, CD32b, and CD32c.

CD32a is known to be implicated in mediating bacterial-activated platelet responses and to play an important role in platelet activation, adhesion and aggregation in response to injured blood vessels. Moreover, CD32a on platelets plays a role in heparin-induced thrombocytopenia which is a life-threatening side-effect of the heparin therapy.

Furthermore, it is known that CD32a activation is necessary and sufficient to produce T-cell anti-tumor cellular immunity. CD32a is also linked to autoimmunity and allergy. It is for example known, that CD32a induces anaphylactic and allergic reactions. In addition, it is also known that the SNP H131R of the CD32a receptor is coupled with various effects. CD32a is associated with several human autoimmune diseases, such as rheumatoid arthritis and increased risk of infection.

Thus, detecting and targeting CD32a has become of high relevance. Nevertheless, the homology between the major CD32 subtypes is extremely high and therefore, making differentiation between the different types is very difficult. In the field of HIV-research, CD32a was recently identified as potential HIV-reservoir marker, not without controversial discussion due to the technical challenging of identifying a small subset of CD32a-positive cells in the presence of CD32b. A sequential, multiple-round, magnetic bead cell-sorting approach had to be used to discriminate between the different CD32-positive immune cell subsets and to isolate the minor population of CD4+CD32+T lymphocytes from peripheral blood due to the lack of a reliable CD32a specific cellular detection reagent.

On genomic level, commercial qPCR primer pairs are available to discriminate between CD32a and CD32b. However, on cellular level and more specifically to detect CD32a on the cellular surface, binding proteins are required, which are able to discriminate between the highly homologous extracellular domains of CD32a and CD32b. Several CD32a antibodies are on the market, however most do also bind CD32b. Those that can discriminate are either raised against the intracellular part of the protein or are not validated for their specificity with regard to detecting CD32a on the cell surface. Taken together there is no known antibody or antibody analogue, which can selectively bind CD32a on the surface of cells.

The present inventors have designed binding proteins, which enable the selective recognition of CD32a positive cells in a mixture of CD32a and CD32b expressing cells without prior depletion of the CD32b positive cell population. The discovered binding proteins specifically bind to the extracellular domain of the CD32a receptor.

### SUMMARY OF THE INVENTION

In a first aspect, the present invention provides a binding protein comprising at least one ankyrin repeat domain, wherein said at least one ankyrin repeat domain specifically binds CD32a, and wherein said at least one ankyrin repeat domain comprises an ankyrin repeat module having an amino acid sequence selected from the group consisting of
i) SEQ ID: 28, 29 and 30, and
ii) SEQ ID NO: 28, 29 and 30 independent of each other comprising up to 10 amino acid substitutions,
preferably wherein the substitutions are independent of each other at positions 1, 3, 4, 6, 14, 15, and/or 27 of SEQ ID NO: 28, 29 and 30 and/or independent of each other at up to three other positions of SEQ ID NO: 28, 29 and 30.

In a second aspect, the invention relates to a binding protein comprising at least one ankyrin repeat domain, wherein said at least one ankyrin repeat domain specifically binds CD32a, and wherein said at least one ankyrin repeat domain comprises an amino acid sequence that has at least 70% amino acid sequence identity with one ankyrin repeat domain selected from the group consisting of SEQ ID NOs: 1-27.

In a third aspect, the invention relates to a nucleic acid comprising a nucleic acid sequence encoding a binding protein, the N-terminal capping repeats and/or the C-terminal capping repeats described herein.

In a further aspect, the invention relates to a host cell comprising the nucleic acid of the third aspect, wherein it optionally expresses a binding protein of the invention.

In a further aspect, the invention relates to a vector comprising the nucleic acid molecule of the third aspect.

In further aspect, the invention relates to a particle comprising a molecule, wherein the molecule is optionally a binding protein herein described.

In a further aspect, the invention relates to a composition comprising the binding protein described herein, the nucleic acid described herein, the host cell described herein, the vector described herein, the particle described herein, or a plurality thereof.

In further aspect, the invention relates to pharmaceutical composition comprising at least one of: the binding protein, the nucleic acid, the host cell, the vector, and/or the particle described herein.

In further aspect, the invention relates to the mentioned binding protein, nucleic acid, host cell, vector, particle, composition or pharmaceutical composition for use as a medicament.

In yet another aspect, the invention relates to a kit comprising the mentioned binding protein, nucleic acid, vector, host cell, particle, composition or pharmaceutical composition

In one aspect of the invention, the herein disclosed binding proteins, nucleic acids, vectors, particles, host cells, compositions, pharmaceutical compositions, or the kit are for use in the treatment, prevention or diagnosis of a condition.

### LIST OF FIGURES

In the following, the content of the figures comprised in this specification is described. In this context please also refer to the detailed description of the invention above and/or below.

FIG. 1: Identification of DARPin Clones Binding to Cell-Surface-Exposed CD32a. DARPin crude E. coli extracts were analysed for binding to stable expressing CD32 Sup T1 cells (SupT1-CD32a) or CD32b SupT1 cells (SupT1-CD32b) for N3C DARPins and on stable expressing CD32 CHO cells (CHO-CD32a or CHO-CD32b) for N2C DARPins via flow cytometry. Two DARPins which recognized CD32a as well as CD32b on the cell surface are 53.F1 and 53.2.D8. A: CD32a expressing cell line; B: CD32b expressing cell line.

FIG. 2: Sequence analysis of the identified N3C DARPins. Sequence logo of the generated DARPin library covering the N Cap, the Repeat elements and the C Cap. To verify the frame work (grey) and diversified positions (black) of the generated N3C DARPin library, the output repertoire was cloned into the plasmid pQE-HisHA and transformed into E. coli XL 1 Blue. Out of 33 clones derived from the VV-N3C library, 26 sequences encoded a unique DARPin and were used to create the sequence logo using WebLogo 3 (http://weblogo.threeplusone.com/create.cgi). The DARPin 52.H6 derived from the VV-N2C library is not included in this analysis.

FIG. 3: Specific labelling of CD32a-positive cell populations with purified CD32a-DARPins. Purified DARPins were analyzed for binding to stable expressing CD32a CHO cells (CHO-CD32a), CD32b CHO cells (CHO-CD32b) or wild type CHO via flow cytometry.

FIG. 4: Protein sequence alignment of the extracellular domains of CD32a, CD32b und CD32c isotypes. An alignment of the extracellular domain of human CD32a (amino acids 34 to 217 of SEQ ID NO: 87), wherein the histidine at position 167 is mutated to arginine, which is also referred to as “CD32a H167R” with reference to the full length CD32a or as “mCD32a H134R” with reference to the mature CD32a), the extracellular domain of human CD32b (amino acids 43 to 217 of SEQ ID NO: 88) and the extracellular domain of human CD32c (amino acids 43 to 223 of SEQ ID NO: 89). Differences in the amino acid sequences are highlighted in grey. The underlined histidine at position 134 (corresponding to amino acids 167 in the full-length sequence CD32a) is not considered relevant for the specific binding, as for the ribosomal display procedure recombinant CD32a protein was used harboring the H167R mutation and in the cellular binding assays cells overexpressing the wild-type H167 CD32a protein.

FIG. 5: Binding of DARPins to chimeric SupT1-CD32b cell lines. Purified DARPins were analyzed for binding to chimeric CD32b proteins as well as the wild type CD32b or CD32a protein stable expressed on CHO cells via flow cytometry compared to secondary antibody control staining. An overview of mutations and their position in the ectodomain of CD32b can be found in Table 2.

FIG. 6: CD32a-specific gene transfer by LVs displaying CD32a-DARPins. Binding of recombinant CD32 protein to selected CD32a-DARPins fused to NiV-G protein transiently expressed on the cell surface of 293T cells.

FIG. 7: Schematic composition of a Nipah virus pseudotyped lentiviral vector (modified, source: Frank et al, 2018).

FIG. 8: CD32a-specific DARPin transduction. Identification of CD32a-specific DARPins mediating transduction by LV vector particles produced in a small-scale approach without concentration.

FIG. 9: Transduction by a Nipah pseudotyped CD32a. 53.2.F11-Lentiviral vector produced in large scale production comparing FCS and Panexin as cell culture supplement.

FIG. 10: Affinity determination of the purified DARPin 53.2.F11[TEV] to human CD32a and CD32b. Sensograms of the DARPin binding with varying concentrations to immobilized recombinant human CD32a target protein [A] or recombinant human off-target CD32b protein [B]. Peaks caused by buffer inequalities during injections were removed manually.

### DETAILED DESCRIPTION OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Klbl, H. eds. (1995), Helvetica Chimica Acta, CH-4010 Basel, Switzerland) and as described in “Pharmaceutical Substances: Syntheses, Patents, Applications” by Axel Kleemann and Jurgen Engel, Thieme Medical Publishing, 1999; the “Merck Index: An Encyclopedia of Chemicals, Drugs, and Biologicals”, edited by Susan Budavari et al., CRC Press, 1996, and the United States Pharmacopeia-25/National Formulary-20, published by the United States Pharmacopeial Convention, Inc., Rockville Md., 2001.

The practice of the present invention will employ, unless otherwise indicated, conventional methods of chemistry, biochemistry, cell biology, immunology. and recombinant DNA techniques which are explained in the literature in the field (cf., e.g., Molecular Cloning: A Laboratory Manual, 2^{nd} Edition, J. Sambrook et al. eds., Cold Spring Harbor Laboratory Press, Cold Spring Harbor 1989).

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. In the following passages, different aspects of the invention are defined in more detail. Each aspect so defined may be combined with any other aspect or aspects unless clearly indicated to the contrary. In particular, any feature indicated as being optional, preferred or advantageous may be combined with any other feature or features indicated as being optional, preferred or advantageous.

In the following, the elements of the present invention will be described. These elements are listed with specific embodiments; however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

### Definitions

In the following, some definitions of terms frequently used in this specification are provided. These terms will, in each instance of its use, in the remainder of the specification have the respectively defined meaning and preferred meanings.

As used in this specification and the appended claims, the singular forms “a”, “an”, and “the” include plural referents, unless the content clearly dictates otherwise. Recitation of ranges of values herein is merely intended to serve as a shorthand method of referring individually to each separate value falling within the range. Unless otherwise indicated herein, each individual value is incorporated into the specification as if it was individually recited herein. All method described herein can be performed in any suitable order unless otherwise indicated herein or otherwise clearly contradicted by context. The use of any and all examples, or exemplary language (e.g., “such as”), provided herein is intended merely to better illustrate the disclosure and does not pose a limitation on the scope of the claims. No language in the specification should be construed as indicating any non-claimed element essential to the practice of the disclosure.

The term “about” means approximately or nearly, and in the context of a numerical value or range set forth herein in one embodiment means ±20%, ±10%, ±5%, or ±3% of the numerical value or range recited or claimed.

Unless expressly specified otherwise, the term “comprising” is used in the context of the present document to indicate that further members may optionally be present in addition to the members of the list introduced by “comprising”. It is, however, contemplated as a specific embodiment of the present disclosure that the term “comprising” encompasses the possibility of no further members being present, i.e., for the purpose of this embodiment “comprising” is to be understood as having the meaning of “consisting of”.

The term “protein” refers in the context of the present invention to a polypeptide, wherein at least part of the polypeptide has, or is able to acquire a defined three-dimensional arrangement by forming secondary, tertiary, or quaternary structures within and/or between its polypeptide chain(s). If a protein comprises two or more polypeptides, the individual polypeptide chains may be linked non-covalently or covalently, e.g. by a disulphide bond between two polypeptides. A part or a protein, which individually has, or is able to acquire a define three-dimensional arrangement by forming secondary or tertiary structures, is termed “protein domain”. Such protein domains are well known to the practitioner skilled in the art.

Amino acids are the building blocks that form peptides, polypeptides and proteins. The following shows the abbreviations and single letter codes used for amino acids.

| | Abbreviation | Abbreviation |
|---|---|---|
| Full Name | (3 Letter) | (1 Letter) |
| Alanine | Ala | A |
| Arginine | Arg | R |
| Asparagine | Asn | N |
| Aspartate | Asp | D |
| Aspartate or Asparagine | Asx | B |
| Cysteine | Cys | C |
| Glutamate | Glu | E |
| Glutamine | Gln | Q |
| Glutamate or Glutamine | Glx | Z |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |

According to the disclosure, the term “peptide” comprises oligo- and polypeptides and refers to substances which comprise about two or more, about 3 or more, about 4 or more, about 6 or more, about 8 or more, about 10 or more, about 13 or more, about 16 or more, about 20 or more, and up to about 50, about 100 or about 150, consecutive amino acids linked to one another via peptide bonds. The term “protein” or “polypeptide” refers to large peptides, in particular peptides having at least about 151 amino acids, but the terms “peptide”, “protein” and “polypeptide” are used herein usually as synonyms.

In the context of the present invention, the term “polypeptide” relates to a molecule consisting of one or more chains of multiple, i.e. two or more, amino acids linked via peptide bonds. Preferably, a polypeptide consists of more than eight amino acids linked via peptide bonds.

For the purposes of the present invention, “variants” of an amino acid sequence (peptide, protein or polypeptide) comprise amino acid insertion variants, amino acid addition variants, amino acid deletion variants and/or amino acid substitution variants. The term “variant” includes all splice variants, posttranslationally modified variants, conformations, isoforms and species homologs, in particular those which are naturally expressed by cells. The term “variant” includes, in particular, fragments of an amino acid sequence.

Amino acid insertion variants comprise insertions of single or two or more amino acids in a particular amino acid sequence. In the case of amino acid sequence variants having an insertion, one or more amino acid residues are inserted into a particular site in an amino acid sequence, although random insertion with appropriate screening of the resulting product is also possible. Amino acid addition variants comprise amino- and/or carboxy-terminal fusions of one or more amino acids, such as 1, 2, 3, 5, 10, 20, 30, 50, or more amino acids. Amino acid deletion variants are characterized by the removal of one or more amino acids from the sequence, such as by removal of 1, 2, 3, 5, 10, 20, 30, 50, or more amino acids. The deletions may be in any position of the protein. Amino acid deletion variants that comprise the deletion at the N-terminal and/or C-terminal end of the protein are also called N-terminal and/or C-terminal truncation variants. Amino acid substitution variants are characterized by at least one residue in the sequence being removed and another residue being inserted in its place. Preference is given to the modifications being in positions in the amino acid sequence which are not conserved between homologous proteins or peptides and/or to replacing amino acids with other ones having similar properties. Preferably, amino acid changes in peptide and protein variants are conservative amino acid changes, i.e., substitutions of similarly charged or uncharged amino acids. A conservative amino acid change involves substitution of one of a family of amino acids which are related in their side chains.

Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cysteine, serine, threonine, tyrosine) amino acids. Phenylalanine, tryptophan, and tyrosine are sometimes classified jointly as aromatic amino acids. In one embodiment, conservative amino acid substitutions include substitutions within the following groups: glycine, alanine; valine, isoleucine, leucine; aspartic acid, glutamic acid; asparagine, glutamine; serine, threonine; lysine, arginine; and phenylalanine, tyrosine.

The term “sequence similarity” as used in the context of the present invention indicates the percentage of amino acids that either are identical or that represent conservative amino acid exchanges. The term “Sequence identity” between two amino acid sequences indicates the percentage of amino acids that are identical between two given sequences. The term “percentage identity” is intended to denote a percentage of amino acid residues which are identical between the two sequences to be compared, obtained after the best alignment, this percentage being purely statistical and the differences between the two sequences being distributed randomly and over their entire length. Sequence comparisons between two amino acid sequences are conventionally carried out by comparing these sequences after having aligned them optimally, said comparison being carried out by segment or by “window of comparison” in order to identify and compare local regions of sequence similarity. The alignment for determining sequence similarity, preferably sequence identity, can be done with art known tools, preferably using the best sequence alignment, for example, using CLC main Workbench (CLC bio) or Align, using standard settings, preferably EMBOSS::needle, Matrix: Blosum62, Gap Open 10.0, Gap Extend 0.5. The percentage of identity is determined with reference to the full-length sequence that is used for comparison and not just for the sequence or sequence stretch with the highest similarity. Thus, an amino acid that shares 100% sequence identity to 50 consecutive amino acids of the 100 amino acid long sequence that is used for comparison only has 50% sequence identity (on the assumption that there are no further amino acids that share any identity outside the 50 consecutive amino acids.

Homologous amino acid sequences exhibit according to the disclosure at least 40%, in particular at least 50%, at least 60%, at least 70%, at least 80%, at least 90% and preferably at least 95%, at least 98 or at least 99% identity of the amino acid residues.

Preferably the degree of similarity, preferably identity between a given amino acid sequence and an amino acid sequence which is a variant of said given amino acid sequence will be in the context of the binding proteins of the present invention at least about 60%, 65%, 70%, 80%, 81%, 82%, 83%, 84%, 85%, 86%, 87%, 88%, 89%, 90%, 91%, 92%, 93%, 94%, 95%, 96%, 97%, 98%, or 99%. The degree of similarity or identity is given preferably for an amino acid region which is at least about 10%, at least about 20%, at least about 30%, at least about 40%, at least about 50%, at least about 60%, at least about 70%, at least about 80%, at least about 90% or about 100% of the entire length of the reference amino acid sequence. For example, if the reference amino acid sequence consists of 200 amino acids, the degree of similarity or identity is given preferably for at least about 20, at least about 40, at least about 60, at least about 80, at least about 100, at least about 120, at least about 140, at least about 160, at least about 180, or about 200 amino acids, preferably continuous amino acids. In preferred embodiments, the degree of similarity or identity is given for the entire length of the reference amino acid sequence. The alignment for determining sequence similarity, preferably sequence identity can be done with art known tools, preferably using the best sequence alignment, for example, using Align, using standard settings, preferably EMBOSS::needle, Matrix: Blosum62, Gap Open 10.0, Gap Extend 0.5.

The amino acid sequence variants described herein may readily be prepared by the skilled person, for example, by recombinant DNA manipulation. The manipulation of DNA sequences for preparing peptides or proteins having substitutions, additions, insertions or deletions, is described in detail in Sambrook et al. (1989), for example. Furthermore, the peptides and amino acid variants described herein may be readily prepared with the aid of known peptide synthesis techniques such as, for example, by solid phase synthesis and similar methods.

In one embodiment, a fragment or variant of an amino acid sequence (peptide or protein) is preferably a “functional fragment” or “functional variant”. The term “functional fragment” or “functional variant” of an amino acid sequence as used in the context of the present invention relates to any fragment or variant exhibiting one or more functional properties identical or similar to those of the amino acid sequence from which it is derived, i.e., it is functionally equivalent. The term “functional fragment” or “functional variant”, as used herein, in particular refers to a variant molecule or sequence that comprises an amino acid sequence that is altered by one or more amino acids compared to the amino acid sequence of the parent molecule or sequence and that is still capable of fulfilling one or more of the functions of the parent molecule or sequence, e.g., binding to a target molecule. In one embodiment, the modifications in the amino acid sequence of the parent molecule or sequence do not significantly affect or alter the binding characteristics of the molecule or sequence. In different embodiments, binding of the functional fragment or functional variant may be reduced but still significantly present, e.g., binding of the functional variant may be at least 50%, at least 60%, at least 70%, at least 80%, or at least 90% of the parent molecule or sequence. However, in other embodiments, binding of the functional fragment or functional variant may be enhanced compared to the parent molecule or sequence.

An amino acid sequence (peptide, protein or polypeptide) “derived from” a designated amino acid sequence (peptide, protein or polypeptide) refers to the origin of the first amino acid sequence. Preferably, the amino acid sequence which is derived from a particular amino acid sequence has an amino acid sequence that is identical, essentially identical or homologous to that particular sequence or a fragment thereof. Amino acid sequences derived from a particular amino acid sequence may be variants of that particular sequence or a fragment thereof. For example, it will be understood by one of ordinary skill in the art that the antigens suitable for use herein may be altered such that they vary in sequence from the naturally occurring or native sequences from which they were derived, while retaining the desirable activity of the native sequences.

The term “recombinant” as used in recombinant protein, recombinant protein domain, recombinant binding protein and the like, means that said polypeptides are produced by the use of recombinant DNA technologies well known by the practitioner skilled in the relevant art. For example, a recombinant DNA molecule (e.g. produced by gene synthesis) encoding a polypeptide can be cloned into a bacterial expression plasmid (e.g. pQE30, Qiagen), yeast expression plasmid or mammalian expression plasmid. When, for example, such a constructed recombinant bacterial expression plasmid is inserted into an appropriate bacteria (e.g. Escherichia coli), this bacteria can produce the polypeptide encoded by this recombinant DNA. The correspondingly produced polypeptide is called a recombinant polypeptide. Thus, the term “recombinant” in the context of the present invention means “made through genetic engineering”. Preferably, a “recombinant object” such as a recombinant cell in the context of the present invention is not occurring naturally.

The term “naturally occurring” as used herein refers to the fact that an molecule can be found in nature. For example, a peptide or nucleic acid that is present in an organism (including viruses) and can be isolated from a source in nature and which has not been intentionally modified by man in the laboratory is naturally occurring.

The term “binding protein” refers in the context of the present invention to a protein comprising one or more binding domains. Furthermore, any such binding protein may comprise additional protein domains that are not binding domains, multimerization moieties, polypeptide tags, polypeptide linkers and/or non-proteinaceous polymer molecules. Examples of multimerization moieties are immunoglobulin heavy chain constant regions which pair to provide functional immunoglobulin Fc domains, and leucine zippers or polypeptides comprising a free thiol which forms an intermolecular disulphide bond between two such polypeptides. Examples of non-proteinaceous polymer molecules are hydroxyethyl starch (HES), polyethylene glycol (PEG), polypropylene glycol, or polyoxyalkylene. The single Cys residue may be used for conjugating other moieties to the polypeptide, for example, by using the maleimide chemistry well known to the person skilled in the art. Preferably, said binding protein is a recombinant binding protein.

The term “repeat proteins” refers to a protein/(poly)peptide comprising one or more repeat domains. Preferably, each of said repeat proteins comprises up to four repeat domains. More preferably, each of said repeat proteins comprises up to two repeat domains. Most preferably, each of the repeat proteins comprises only one repeat domain. Furthermore, said repeat protein may comprise additional non-repeat protein domains, (poly)peptide tags, (poly)peptide linkers, enzymes (for example alkaline phosphatase) which may allow the detection of repeat proteins, or moieties which can be used for targeting (such as immunoglobulins or fragments thereof) and/or effector molecules.

The term “(poly)peptide tag” or “protein tag” interchangeably refer in the context of the present invention to an amino acid sequence attached to a (poly)peptide/protein, wherein said amino acid sequence is useful for the purification, detection, or targeting of said (poly)peptide/protein, or wherein said amino acid sequence improves the physicochemical behavior of the polypeptide/protein, or wherein said amino acid sequence possesses an effector function. The individual (poly)peptide tags, moieties and/or domains of a binding protein may be connected to each other directly or via polypeptide linkers. These polypeptide tags are all well known in the art and are fully available to the person skilled in the art. Examples of polypeptide tags are small polypeptide sequences, for example, His, HA, myc, FLAG, or Strep-tags, GFP, or moieties such as enzymes (for example enzymes like alkaline phosphatase), which allow the detection of said (poly)peptide/protein, or moieties which can be used for targeting (such as immunoglobulins or fragments thereof) and/or as effector molecules. An enzyme may be also a protease, such as a TEV protease, the enzyme may also be for example factor X. The (poly)peptides or proteins may also present a tag such as a fluorophore tag or a radiolabeling tag, largely known in the art.

The term “polypeptide linker” refers in the context of the present invention to an amino acid sequence, which is able to link, for example, two protein domains, a polypeptide tag and a protein domain, a protein domain and a non-polypeptide moiety such as polyethylene glycol or two sequence tags. Such additional domains, tags, non-polypeptide moieties and linkers are known to the person skilled in the relevant art. Particular examples of such linkers are glycine-serine-linkers and proline-threonine-linkers of variable lengths; preferably, said linkers have a length between 2 and 24 amino acids; more preferably, said linkers have a length between 2 and 16 amino acids.

The term “repeat domain” refers in the context of the present invention to a protein domain comprising two or more consecutive repeat units (modules) as structural units, wherein said structural units have the same fold, and stack tightly to create, for example, a superhelical structure having a joint hydrophobic core. Preferably, a repeat domain further comprises an N-terminal and/or a C-terminal capping unit (or module). Even more preferably, said N-terminal and/or C-terminal capping units (or modules) are capping repeats.

A repeat protein may further comprise an N- and/or a C-terminal capping module having an amino acid sequence different from any one of said repeat modules. The term “capping module” refers to a polypeptide fused to the N- or C-terminal repeat module of a repeat domain, wherein said capping module forms tight tertiary interactions with said repeat module thereby providing a cap that shields the hydrophobic core of said repeat module at the side not in contact with the consecutive repeat module from the solvent. Said N- and/or C-terminal capping module may be, or may be derived from, a capping unit or other domain found in a naturally occurring repeat protein adjacent to a repeat unit.

The term “capping unit” refers in the context of the present invention to naturally occurring folded (poly)peptide, wherein said (poly)peptide defines a particular structural unit which is N- or C-terminally fused to a repeat unit, wherein said (poly)peptide forms tight tertiary interactions with said repeat unit thereby providing a cap that shields the hydrophobic core of said repeat unit at one side from the solvent. Preferably, capping units are capping repeats. The term “capping repeat” refers to capping unit having a similar or the same fold as said adjacent repeat unit and/or sequence similarities to said adjacent repeat unit.

The term “structural unit” refers in the context of the present invention to a locally ordered part of a polypeptide, formed by three-dimensional interactions between two or more segments of secondary structure that are near one another along the polypeptide chain. Such a structural unit exhibits a structural motif. The term “structural motif” refers to a three-dimensional arrangement of secondary structure elements present in at least one structural unit. Structural motifs are well known to the person skilled in the art. Structural units alone are not able to acquire a defined three-dimensional arrangement; however, their consecutive arrangement, for example as repeat modules in a repeat domain, leads to a mutual stabilization of neighboring units resulting in a superhelical structure.

The term “designed repeat protein” and “designed repeat domain” interchangeably refer in the context of the present invention to a repeat protein or repeat domain, respectively. Designed repeat proteins and designed repeat domains are synthetic and not from nature. They are man-made proteins or domains, respectively, obtained by expression of correspondingly designed nucleic acids. Preferably, the expression is done in eukaryotic or prokaryotic cells, such as bacterial cells, or by using a cell-free in vitro expression system. Accordingly, a designed ankyrin repeat protein (i.e. a DARPin) corresponds to a binding protein of the invention comprising at least one ankyrin repeat domain.

The term “repeat unit” refers in the context of the present invention to the repeated amino acid sequences of the designed repeat domains, which are originally derived from the repeat units (modules) of naturally occurring repeat proteins. Each repeat domain is derived from one or more repeat units of the family or subfamily or naturally occurring repeat proteins, e.g. the family or armadillo repeat proteins or ankyrin repeat proteins.

The term “repeat modules” refers in the context of the present invention to amino acid sequences comprising repeat sequence motifs of one or more naturally occurring repeat proteins, wherein said “repeat modules” (units) are found in multiple copies, and which exhibit a defined folding topology common to all said motifs determining the fold of the protein. Such repeat units comprise framework residues and interaction residues. Examples of such repeat units are armadillo repeat units, leucine rich repeat units, ankyrin repeat units, tetratricopeptide repeat units, HEAT repeat units, and leucine-rich variant repeat units. Naturally occurring proteins containing two or more such repeat units are referred to as “naturally occurring repeat proteins”. The amino acid sequences of the individual repeat units of a repeat protein may have a significant number of mutations, substitutions, additions and/or deletions when compared to each other, while still substantially retaining the general pattern, or motif, of the repeat units.

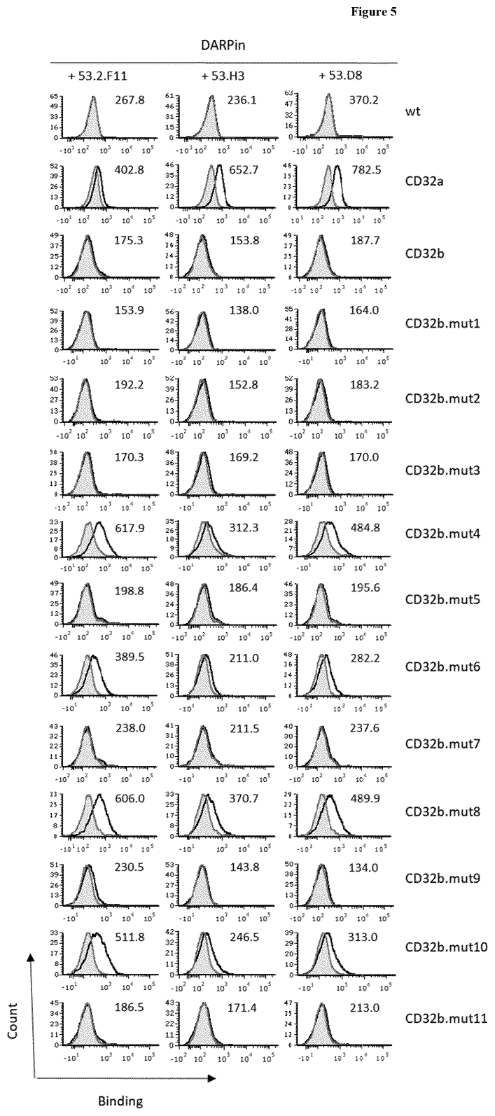

The term “set of repeat modules” refers in the context of the present invention to the total number of repeat modules present in a repeat domain. Such “set of repeat modules” present in a repeat domain comprises two or more consecutive repeat modules, and may comprise just one type of repeat module in two or more copies, or two or more different types of modules, each present in one or more copies. Such set of repeat modules comprising, for example, 3 repeat modules may comprise consecutively, form N- to C-terminus, repeat module 1, repeat module 2, and repeat module 3, as shown for example, in FIG. 5. Repeat module 1 as shown in FIG. 5 preferably comprises amino acids 29 to 61. Repeat module 2 as shown in FIG. 5 preferably comprises amino acids 62 to 94. Repeat module 3 as shown in FIG. 5 preferably comprises amino acids 95 to 127.

Different repeat domains may have an identical number of repeat modules per repeat domain or may differ in the number of repeat modules per repeat domain.

Preferably, the repeat modules comprised in a set are homologous repeat modules. In the context of the present invention, the term “homologous repeat modules” refers to repeat modules, wherein more than 70% of the framework residues of said repeat modules are homologous. Preferably, more than 80% of the framework residues of said repeat modules are homologous. Most preferably, more than 90% of the framework residues of said repeat modules are homologous. Computer programs to determine the percentage of homology between polypeptides, such as Fasta, Blast or Gap, are known to the person skilled in the relevant art.

Repeat units may comprise positions with amino acid residues present in all copies of corresponding repeat units (“fixed positions”) and positions with differing or “randomized” amino acid residues (“randomized positions”), i.e. framework residues and interaction residues.

One example of such repeat units is an ankyrin repeat unit. Naturally occurring proteins containing two or more such repeat units are referred to as “naturally occurring repeat proteins”. The amino acid sequences of the individual repeat units of a repeat protein may have a significant number of mutations, substitutions, additions and/or deletions when compared to each other, while still substantially retaining the general pattern, or motif, of the repeat units.

The term “ankyrin repeat unit” refers in the context of the present invention to a repeat unit, which is an ankyrin repeat. Ankyrin repeats are well known to the person skilled in the art.

The term “DARPin” refers in the context of the present invention to designed ankyrin repeat proteins that are genetically engineered antibody mimetic proteins exhibiting highly specific and high-affinity target protein binding. DARPins are well known in the art and were first described by Binz H K, et al. (2003) JMB. 332 (2): 489-503 and are reviewed in Plickthun A (2015). Annu. Rev. Pharmacol. Toxicol. 55 (1): 489-511. DARPins are based on naturally occurring ankyrin repeat proteins, yet contain one or more amino acid mutations that can affect, for example, their binding affinity to a given target molecule, their cell surface expression, and the like. Typically, DARPins comprise four or five repeats, of which the first (N-capping repeat) and last (C-capping repeat) serve to shield the hydrophobic protein core from the aqueous environment. DARPins correspond to the average size of natural ankyrin repeat protein domains. Proteins with fewer than three repeats (i.e., the capping repeats and one internal repeat) do not form a stable enough tertiary structure. The molecular mass of a DARPin depends on the total number of repeats, as shown in the following chart: DARPins of the present invention preferably include 2 to 6, preferably 3 to 5 ankyrin repeat units flanked by N- and C-capping repeats. Each ankyrin repeat unit includes about 33 amino acid residues.

The term “repeat sequence motif” or “repeat consensus sequence” refers in the context of the present invention to an amino acid sequence, which is deduced from one or more repeat units. Such repeat sequence motifs comprise framework residue positions and target interaction residue positions. Said framework residue positions correspond to the positions of framework residues of said repeat units. Said target interaction residue positions correspond to the positions of target interaction residues of said repeat units. Such repeat sequence motifs comprise fixed positions and randomized positions. The term “fixed position” refers to an amino acid position in a repeat sequence motif, wherein said position is set to a particular amino acid. Frequently, such fixed positions correspond to the positions of framework residues.

The term “randomized position” refers in the context of the present invention to an amino acid position in a repeat sequence motif, wherein two or more amino acids are allowed at said amino acid position. Frequently, such randomized positions correspond to the positions of target interaction residues. However, some positions of framework residues may also be randomized.

The term “framework residues” refers in the context of the present invention to amino acid residues of the repeat units, or the corresponding amino acid residues of the repeat modules, which contribute to the folding topology, i.e. which contribute to the fold of said repeat unit (or module) or which contribute to the interaction with a neighboring unit (or module). Such contribution might be the interaction with other residues in the repeat unit (or module), or the influence on the polypeptide backbone conformation as found in α-helices or β-sheets, or amino acid stretches forming linear polypeptides or loops.

The term “target interaction residues” refers in the context of the present invention to amino acid residues of the repeat units, or the corresponding amino acid residues of the repeat modules, which contribute to the interaction with target substances. Such contribution might be the direct interaction with the target substances, or the influence on other directly interacting residues, e.g. by stabilizing the conformation of the polypeptide of a repeat unit (or module) to allow or enhance the interaction of directly interacting residues with said target. Such framework and target interaction residues may be identified by analysis of the structural data obtained by physicochemical methods, such as X-ray crystallography, NMR and/or CD spectroscopy, or by comparison with known and related structural information well known to practitioners in structural biology and/or bioinformatics.

The term “folding topology” refers in the context of the present invention to the tertiary structure of said repeat units or repeat modules. The folding topology will be determined by stretches of amino acids forming at least parts of α-helices or β-sheets, or amino acid stretches forming linear polypeptides or loops, or any combination of α-helices, β-sheets and/or linear polypeptides/loops.

The term “consecutive” refers in the context of the present invention to an arrangement, wherein the repeat units or repeat modules are arranged in tandem. In designed repeat proteins, there are at least 2, usually about 2 to 6, in particular at least about 6, frequently 20 or more repeat units. In most cases, repeat units of a repeat domain will exhibit a high degree of sequence identity (same amino acid residues at corresponding positions) or sequence similarity (amino acid residues being different, but having similar physicochemical properties), and some of the amino acid residues might be key residues being strongly conserved. However, a high degree of sequence variability by amino acid insertions and/or deletions, and/or substitutions between the different repeat units of a repeat domain may be possible as long as the common folding topology of the repeat units is maintained.

In most cases, said repeat units will exhibit a high degree of sequence identity (same amino acid residues at corresponding positions) or sequence similarity (amino acid residues being different, but having similar physicochemical properties), and some of the amino acid residues might be key residues being strongly conserved in the different repeat units found in naturally occurring proteins.

However, a high degree of sequence variability by amino acid insertions and/or deletions, and/or substitutions between the different repeat units found in naturally occurring proteins will be possible as long as the common folding topology is maintained.

The terms “specifically binding to a target”, “target specificity”, “has binding specificity for a target”, “specific binding” or “specifically binding” interchangeably mean in the context of the present invention that a binding protein or binding domain binds to the extracellular domain of human CD32a (amino acids 34 to 217 of SEQ ID NO: 87 (optionally to the extracellular domain of the natural CD32a variants Q63R, M140V, H167R and I218V, the positions are indicated with reference to the full length sequence according to SEQ ID NO: 87) with a lower dissociation constant than to an unrelated protein, preferably a binding protein or binding domain of the present invention binds to the extracellular domain of human CD32a (amino acids 34 to 217 of SEQ ID NO: 87 (optionally to the extracellular domain of the natural CD32a variants Q63R, M140V, H167R and I218V) with a lower dissociation constant than to the extracellular domain of human CD32b (amino acids 43 to 217 of SEQ ID NO:88) or to the extracellular domain of human CD32c (amino acids 43 to 223 of SEQ ID NO: 89). A lower dissociation constant may be at least 10-fold lower, preferably 10²-fold lower, more preferably 10³-fold lower, more preferably 10⁴-fold lower, and even more preferably 10⁵-fold lower. In a most preferred embodiment, the dissociation constant for binding to the extracellular domain of human CD32a (amino acids 34 to 217 of SEQ ID NO: 87 (optionally to the natural variants Q63R, M140V, H167R and I218V is at least 10⁶, more preferably 107, even more preferably 10⁸, or most preferably 109 times lower than the corresponding dissociation constant for the unrelated protein, such as to the extracellular domain of human CD32b (amino acids 43 to 217 of SEQ ID NO: 88) and with a lower dissociation constant than to the extracellular domain of human CD32c (amino acids 43 to 223 of SEQ ID NO: 89).

The term “target” refers to an individual molecule such as a nucleic acid molecule, a polypeptide or protein, a carbohydrate, or any other naturally occurring molecule, including any part of such individual molecule, or complexes of two or more of such molecules. The target may be a whole cell or a tissue sample, or it may be any non-natural molecule or moiety. Preferably, the target is a naturally occurring or non-natural polypeptide or a polypeptide containing chemical modifications, for example modified by natural or non-natural phosphorylation, acetylation, or methylation. In particular, in the present invention, the target is CD32a.

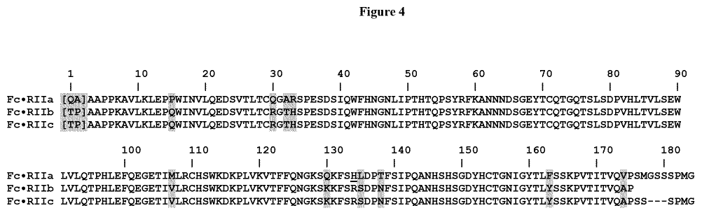

The term “CD32a” refers to a protein that is also known as FCGR2A. CD32a is the low affinity receptor II-a that binds to the Fc region of immunoglobulin gamma. The extracellular domain of human CD32a is highly homologous to the extracellular domains of the related proteins CD32b and CD32c. An alignment of the extracellular domains of human CD32a (amino acids 34 to 217 of SEQ ID NO: 87), wherein the histidine at position 167 is mutated to arginine, which is also referred to as “CD32a H167R” with reference to the full length CD32a or as “mCD32a H134R” with reference to the mature CD32a), the extracellular domain of human CD32b (amino acids 43 to 217 of SEQ ID NO: 88) and the extracellular domain of human CD32c (amino acids 43 to 223 of SEQ ID NO: 89) is shown in FIG. 4.

The term “consensus sequence” as used in the context of the present invention refers to a calculated order of most frequent residues, either nucleotide or amino acid, found at each position in a sequence alignment between two or more sequences. It represents the results of a multiple sequence alignment in which related sequences are compared to each other and similar sequence motifs are calculated. Conserved sequence motifs are depicted as consensus sequences, which indicate identical amino acids, i.e. amino acids identical among the compared sequences, conserved amino acids, i.e. amino acids which vary among the compared amino acid sequence but wherein all amino acids belong to a certain functional or structural group of amino acids, e.g. polar or neutral, and variable amino acids, i.e. amino acids which show no apparent relatedness among the compared sequence. Table 1 depicts an alignment of sequences of repeat units including N- and C-capping units. The alignments were generated either manually, or alternatively the algorithm used to generate the alignments mentioned was Clustal Omega using the default settings. The positions of the seven amino acids that may be substituted in some embodiments are highlighted.

The term “terminal plasma half-life” of a drug such as a binding protein or binding domain of the invention refers to the time required to reach half the plasma concentration of the drug applied to a mammal after reaching pseudoequilibrium. This half-life is not defined as the time required to eliminate half the dose of the drug administered to the mammal.

The term “bioactive compound” refers to a compound that is disease modifying when applied to a mammal having said disease. A bioactive compound may have antagonistic or agonistic properties and can be a proteinaceous bioactive compound or a non-proteinaceous bioactive compound.

The term “compete for binding” means the inability of two different binding domains of the invention to bind simultaneously to the same target, while both are able to bind the same target individually. Thus, such two binding domains compete for binding to said target. Preferably, said two competing binding domains bind to an overlapping or the same binding epitope on said target. Methods, such as competition Enzyme-Linked Immuno Sorbent Assay (ELISA) or competition SPR measurements (e.g. by using the Proteon instrument from BioRad), to determine if two binding domains compete for binding to a target, are well known to the practitioner in the art.

The term “polynucleotide” or “nucleic acid”, as used herein includes DNA and RNA such as genomic DNA, cDNA, mRNA, recombinantly produced and chemically synthesized molecules. A nucleic acid may be single-stranded or double-stranded. RNA includes in vitro transcribed RNA (IVT RNA) or synthetic RNA. According to the invention, a polynucleotide is preferably isolated. Nucleic acids may be comprised in a vector.

The terms “vector” or “expression vector” are used interchangeably in the context of the present invention to refer to a polynucleotide or a mixture of a polynucleotide and proteins capable of being introduced or of introducing the collection of nucleic acids of the present invention or one nucleic acid that is part of the collection of nucleic acids of the invention into a cell, preferably a mammalian cell. Examples of vectors include but are not limited to plasmid vectors, cosmid vectors, phage vectors such as lambda phage, viral vectors such as retroviral, adenovirus-associated viral vectors (AAV), adenoviral or baculoviral vectors, or artificial chromosome vectors such as bacterial artificial chromosomes (BAC), yeast artificial chromosomes (YAC), or P1 artificial chromosomes (PAC). Said vectors include expression as well as cloning vectors. In particular, a vector is used to transport the promoter and the collection of the nucleic acids or one nucleic acid that is part of the collection of nucleic acids of the invention into a suitable host cell. Expression vectors may contain “replicon” polynucleotide sequences that facilitate the autonomous replication of the expression vector in a host cell. Once in the host cell, the expression vector may replicate independently of or coincidental with the host chromosomal DNA, and several copies of the vector and its inserted DNA can be generated. In case that replication incompetent expression vectors are used—which is often the case for safety reasons—the vector may not replicate but merely direct expression of the nucleic acid. Depending on the type of expression vector the expression vector may be lost from the cell, i.e. only transiently expresses the neo-antigens encoded by the nucleic acid or may be stable in the cell. Expression vectors typically contain expression cassettes, i.e. the necessary elements that permit transcription of the nucleic acid into an mRNA molecule.

The term “viral vector” is used in the context of the present invention to refer to a single or double stranded nucleic acid sequence that can assemble into an infectious viral particle. This nucleic acid sequence may be a full or partial viral genome. In the latter case the viral genome preferably comprises one or more heterologous genes. For some viral particles only very short sequences of the viral genome are required to allow assembly of an infectious viral particle. For example, for assembly of an infectious adeno-associated viral particle only a short (about 200 bp long) repeat sequence placed at the 5′ and 3′ of a heterologous nucleic acid of a given length (typically between 4.5. to 5.3 kB for an adeno-associated virus) will allow assembly of infectious adeno-associated viral particles. The minimal nucleic acid sequence for assembly of a given virus are well known. The larger the viral genome and the smaller the minimal viral sequence for assembly of an infectious viral particle, the bigger the heterologous gene(s) can be that can be inserted into the viral vector.

A “lentivirus” as used herein refers to a genus of the Retroviridae family. Lentiviruses are unique among the retroviruses in being able to infect non-dividing cells; they can deliver a significant amount of genetic information into the DIMA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

A “adenovirus-associated viral vector” as used herein refers to a genus of the Dependoparvovirus, which belongs to the family Parvoviridae. The viral vectors can be used for gene therapy, and can infect both dividing and quiescent cells and persist in an extrachromosomal state without integrating into the genome of the host cell. Adeno-associated virus (AAV) is a non-enveloped virus that can be engineered to deliver DNA to target cells. AAV recombinant particles lacking any viral genes and containing DNA sequences of interest for various therapeutic applications can be generated and provide one of the safest strategies for gene therapies.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if a transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or the product of that gene or cDNA.

According to the disclosure, the term “RNA encodes” means that the RNA, if present in the appropriate environment, such as within cells of a target tissue, can direct the assembly of amino acids to produce the peptide or protein it encodes during the process of translation. In one embodiment, RNA is able to interact with the cellular translation machinery allowing translation of the peptide or protein. A cell may produce the encoded peptide or protein intracellularly (e.g. in the cytoplasm and/or in the nucleus), may secrete the encoded peptide or protein, or may produce it on the surface.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence. Expression can be transient or stable. According to the invention, the term expression also includes an “aberrant expression” or “abnormal expression”.

According to the present invention, gene expression level is deemed “altered” when gene expression is increased or decreased 10%, 25%, 50% or more as compared to the control level. Alternatively, an expression level is deemed “increased” or “decreased” when gene expression is increased or decreased by at least 0.1, at least 0.2, at least 1, at least 2, at least 5, or at least 10 or more fold as compared to a control level.

In the context of the present invention, the phrase “control level” refers to a protein expression level detected in a control sample and includes both a “normal control level” and a “diseased control level”. A control level can be a single expression pattern derived from a single reference population or from a plurality of expression patterns. For example, the control level can be a database of expression patterns from previously tested cells. A “normal control level” refers to a level of gene expression detected in a normal, healthy individual or in a population of individuals known not to be suffering from a condition associated with altered CD32a expression (such as a HIV associated disease). A normal individual is one with no clinical symptoms of a condition associated with altered CD32a expression. On the other hand, a “diseased control level” refers to an expression profile of CD32a-associated genes found in a population suffering from a condition associated with altered CD32a expression.

As used herein, the terms “linked,” “fused”, or “fusion” are used interchangeably. These terms refer to the joining together of two or more elements or components or domains.

In the context of the present disclosure, the term “particle” relates to a structured entity formed by molecules or molecule complexes. In one embodiment, the term “particle” relates to a micro- or nanosized structure, such as a micro- or nano-sized compact structure dispersed in a medium. In one embodiment, a particle is a nucleic acid containing particle such as a particle comprising DNA, RNA or a mixture thereof.

Electrostatic interactions between positively charged molecules such as polymers and lipids and negatively charged nucleic acid are involved in particle formation. This results in complexation and spontaneous formation of nucleic acid particles. In one embodiment, a nucleic acid particle is a nanoparticle.

As used in the present disclosure, “nanoparticle” refers to a particle having an average diameter suitable for parenteral administration.

A “nucleic acid particle” can be used to deliver nucleic acid to a target site of interest (e.g., cell, tissue, organ, and the like). A nucleic acid particle may be formed from at least one cationic or cationically ionizable lipid or lipid-like material such as DOTAP, at least one cationic polymer such as protamine, or a mixture thereof and nucleic acid. Nucleic acid particles include lipid nanoparticle (LNP)-based and lipoplex (LPX)-based formulations.

Without intending to be bound by any theory, it is believed that the cationic or cationically ionizable lipid or lipid-like material and the cationic polymer combine together with the nucleic acid to form aggregates, and this aggregation results in colloidally stable particles.

Particles described herein may further comprise at least one lipid or lipid-like material other than a cationic or cationically ionizable lipid or lipid-like material, at least one polymer other than a cationic polymer, or a mixture thereof.

Nucleic acid particles may comprise more than one type of nucleic acid molecules, where the molecular parameters of the nucleic acid molecules may be similar or different from each other, like with respect to molar mass or fundamental structural elements such as molecular architecture, capping, coding regions or other features, Nucleic acid particles described herein may have an average diameter that in one embodiment ranges from about 30 nm to about 1000 nm, from about 50 nm to about 800 nm, from about 70 nm to about 600 nm, from about 90 nm to about 400 nm, or from about 100 nm to about 300 nm.

Nucleic acid particles described herein, e.g. generated by the processes described herein, exhibit a polydispersity index less than about 0.5, less than about 0.4, less than about 0.3, or about 0.2 or less. By way of example, the nucleic acid particles can exhibit a polydispersity index in a range of about 0.1 to about 0.3 or about 0.2 to about 0.3.

Nucleic acid particles described herein can be prepared using a wide range of methods that may involve obtaining a colloid from at least one cationic or cationically ionizable lipid or lipid-like material and/or at least one cationic polymer and mixing the colloid with nucleic acid to obtain nucleic acid particles.

The term “colloid” as used herein relates to a type of homogeneous mixture in which dispersed particles do not settle out. The insoluble particles in the mixture are microscopic, with particle sizes between 1 and 1000 nanometers. The mixture may be termed a colloid or a colloidal suspension. Sometimes the term “colloid” only refers to the particles in the mixture and not the entire suspension.

The term “average diameter” refers to the mean hydrodynamic diameter of particles as measured by dynamic laser light scattering (DLS) with data analysis using the so-called cumulant algorithm, which provides as results the so-called Z_{average} with the dimension of a length, and the polydispersity index (PI), which is dimensionless (Koppel, D., J. Chem. Phys. 57, 1972, pp 4814-4820, ISO 13321). Here “average diameter”, “diameter” or “size” for particles is used synonymously with this value of the Z_{average}.

The “polydispersity index” is preferably calculated based on dynamic light scattering measurements by the so-called cumulant analysis as mentioned in the definition of the “average diameter”.

Under certain prerequisites, it can be taken as a measure of the size distribution of an ensemble of nanoparticles.

Different types of nucleic acid containing particles have been described previously to be suitable for delivery of nucleic acid in particulate form (e.g. Kaczmarek, J. C. et al., 2017, Genome Medicine 9, 60). For non-viral nucleic acid delivery vehicles, nanoparticle encapsulation of nucleic acid physically protects nucleic acid from degradation and, depending on the specific chemistry, can aid in cellular uptake and endosomal escape.

The present disclosure describes particles comprising nucleic acid, at least one cationic or cationically ionizable lipid or lipid-like material, and/or at least one cationic polymer which associate with nucleic acid to form nucleic acid particles and compositions comprising such particles. The nucleic acid particles may comprise nucleic acid which is complexed in different forms by non-covalent interactions to the particle. The particles described herein are not viral particles, in particular infectious viral particles, i.e., they are not able to virally infect cells.

Suitable cationic or cationically ionizable lipids or lipid-like materials and cationic polymers are those that form nucleic acid particles and are included by the term “particle forming components” or “particle forming agents”. The term “particle forming components” or “particle forming agents” relates to any components which associate with nucleic acid to form nucleic acid particles. Such components include any component which can be part of nucleic acid particles.

Cationic polymer given their high degree of chemical flexibility, polymers are commonly used materials for nanoparticle-based delivery. Typically, cationic polymers are used to electrostatically condense the negatively charged nucleic acid into nanoparticles. These positively charged groups often consist of amines that change their state of protonation in the pH range between 5.5 and 7.5, thought to lead to an ion imbalance that results in endosomal rupture. Polymers such as poly-L-lysine, polyamidoamine, protamine and polyethyleneimine, as well as naturally occurring polymers such as chitosan have all been applied to nucleic acid delivery and are suitable as cationic polymers herein. In addition, some investigators have synthesized polymers specifically for nucleic acid delivery. Poly(O-amino esters), in particular, have gained widespread use in nucleic acid delivery owing to their ease of synthesis and biodegradability. Such synthetic polymers are also suitable as cationic polymers herein.

The term “genetic modification” includes the transfection of cells with nucleic acid. The term “transfection” relates to the introduction of nucleic acids, in particular RNA, into a cell. For purposes of the present invention, the term “transfection” also includes the introduction of a nucleic acid into a cell or the uptake of a nucleic acid by such cell, wherein the cell may be present in a subject, e.g., a patient. Thus, according to the present invention, a cell for transfection of a nucleic acid described herein can be present in vitro or in vivo, e.g. the cell can form part of an organ, a tissue and/or an organism of a patient. According to the invention, transfection can be transient or stable. For some applications of transfection, it is sufficient if the transfected genetic material is only transiently expressed. RNA can be transfected into cells to transiently express its coded protein. Since the nucleic acid introduced in the transfection process is usually not integrated into the nuclear genome, the foreign nucleic acid will be diluted through mitosis or degraded. Cells allowing episomal amplification of nucleic acids greatly reduce the rate of dilution. If it is desired that the transfected nucleic acid actually remains in the genome of the cell and its daughter cells, a stable transfection must occur. Such stable transfection can occur if the nucleic acid introduced in the transfection process is integrated into the nuclear genome and can be achieved, for example, by using virus-based systems or transposon-based systems for transfection. Generally, cells that are genetically modified to express an antigen receptor are stably transfected with nucleic acid encoding the antigen receptor, while, generally, nucleic acid encoding antigen is transiently transfected into cells.

The term “pharmaceutical composition” relates to a formulation comprising a therapeutically effective agent, preferably together with pharmaceutically acceptable carrier, diluents and/or excipients. Said pharmaceutical composition is useful for treating, preventing, or reducing the severity of a disease or disorder by administration of said pharmaceutical composition to a subject. A pharmaceutical composition is also known in the art as a pharmaceutical formulation.

The term “pharmaceutically acceptable”, as used herein, refers to the non-toxicity of a material, which, preferably, does not interact with the action of the active agent of the pharmaceutical composition. In particular, “pharmaceutically acceptable” means approved by a regulatory agency of the Federal or a state government or listed in the U.S. Pharmacopoeia, European Pharmacopoeia or other generally recognized pharmacopeia for use in animals, and more particularly in humans.

The term “carrier” refers to an organic or inorganic component, of a natural or synthetic nature, in which the active component is combined in order to facilitate, enhance or enable application. According to the invention, the term “carrier” also includes one or more compatible solid or liquid fillers, diluents or encapsulating substances, which are suitable for administration to a subject. Possible carrier substances (e.g., diluents) are, for example, sterile water, Ringer's solution, Lactated Ringer's solution, physiological saline, bacteriostatic saline (e.g., saline containing 0.9% benzyl alcohol), phosphate-buffered saline (PBS), Hank's solution, fixed oils, polyalkylene glycols, hydrogenated naphthalenes and biocompatible lactide polymers, lactide/glycolide copolymers or polyoxyethylene/polyoxy-propylene copolymers. In one embodiment, the carrier is PBS. The resulting solutions or suspensions are preferably isotonic to the blood of the recipient. Suitable carriers and their formulations are described in greater detail in Remington's Pharmaceutical Sciences, 17th ed., 1985, Mack Publishing Co.

The term “cell” is used in the context of the present invention to refer to a eukaryotic or a prokaryotic cell, such as a bacterial cell, a yeast cell, or a cell of a mammal, preferably a cell of a human, a mouse, a rat, a rabbit, a dog, a monkey, or a cat.

### Aspects of the Invention and Preferred Embodiments

In the following different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

In the work leading to the present invention, it was surprisingly shown that specific binding proteins can discriminate between the highly homologous CD32a and CD32b.

In a first aspect, the present invention provides a binding protein comprising at least one ankyrin repeat domain, wherein said at least one ankyrin repeat domain specifically binds CD32a, and wherein said at least one ankyrin repeat domain comprises at least one ankyrin repeat module having an amino acid sequence selected from the group consisting of
i) SEQ ID: 28, 29 and 30, and
ii) SEQ ID NO: 28, 29 and 30 independent of each other comprising up to 10 amino acid substitutions.

Preferably, up to 9 amino acids, more preferably up to 8 amino acids, more preferably up to 7 amino acids, more preferably up to 6 amino acids, more preferably up to 5 amino acids, more preferably up to 4 amino acids, more preferably up to 3 amino acids, more preferably up to 2 amino acids, more preferably up to 1 amino acid, and most preferably no amino acid in SEQ ID NO: 28, 29 and 30 is substituted.

Wherein, if 10 amino acids are substituted, the up to 10 substituted amino acids are preferably at positions 1, 3, 4, 6, 14, 15, 27 and/or three any further positions of SEQ ID NO: 28, 29 and 30, if 9 amino acids are substituted, the up to 9 substituted amino acids are preferably at positions 1, 3, 4, 6, 14, 15, 27 and/or two any further positions of SEQ ID NO: 28, 29 and 30, if 8 amino acids are substituted, the up to 8 amino acids are preferably at positions 1, 3, 4, 6, 14, 15, 27 and/or one any further position of SEQ ID NO: 28, 29 and 30, if 7 amino acids are substituted, the up to 7 amino acids are preferably at positions 1, 3, 4, 6, 14, 15, 27 of SEQ ID NO: 28, 29 and 30.

Preferably, when amino acids at position 1, 3, 4, 6, 14 and/or 15 are substituted in SEQ ID NO: 28, 29 and/or 30, these amino acids are any amino acid, preferably any amino acid except glycine, proline or cysteine. Preferably, when the amino acid at position 27 is substituted in SEQ ID NO: 28, 29 and/or 30, this amino acid is any amino acid, preferably it is histidine, asparagine or tyrosine.

In one embodiment of the first aspect, the binding protein comprises at least 2 repeat modules, which may be identical or different. In one embodiment of the first aspect, the binding protein comprises between 2 and 20 repeat modules, which may be identical or different. In one embodiment of the first aspect, the binding protein comprises 3 repeat modules, which may be identical or different.

In a preferred embodiment, the binding protein comprises 3 repeat modules wherein repeat module 1 has an amino acid sequence according to SEQ ID NO: 28, repeat module 2 has amino acid sequence according to SEQ ID NO: 29, and repeat module 3 has an amino acid sequence according to SEQ ID NO: 30, preferably wherein 10 amino acids or less in each module are substituted by any amino acid, preferably 9, 8, 7, 6, 5, 4, 3, 2, or 1 amino acids are substituted, most preferably no amino acid is substituted.

In a preferred embodiment, the binding protein comprises 3 repeat modules wherein repeat module 1 has an amino acid sequence according to SEQ ID NO: 28, repeat module 2 has amino acid sequence according to SEQ ID NO: 29, and repeat module 3 has an amino acid sequence according to SEQ ID NO: 30, preferably, wherein 5 amino acids or less in each module are substituted by any amino acid, preferably 4, 3, 2, or 1, most preferably no amino acid is substituted.

A preferred binding protein comprises at least one ankyrin repeat domain, wherein said ankyrin repeat module binds CD32a with a dissociation constant (Kd) of 2×10⁻⁵ M or lower. Preferably, said ankyrin repeat module binds CD32a with a Kd of 2×10⁻⁶ M or lower, more preferably of 2×10⁻⁷ M or lower, of 2×10⁻⁸ M or lower, or most preferably of 2×10⁻⁹ M or lower if measured by surface plasmon resonance (SPR). Binding proteins comprising an ankyrin repeat domain binding CD32a with a Kd of 2×10⁻⁵ M or lower are shown in the Examples.

It is understood by a skilled person that including two or more repeat domains that bind CD32a in the binding protein of the protein of the present invention can increase the affinity of the binding protein of the present invention due to avidity effects that result from the simultaneous binding of a binding protein of the present invention to two or more CD32a antigens, e.ge. at the surface of a cell. Thus, a preferred binding protein of the present invention comprising one of said repeat domains which binds CD32a will bind to CD32a with a Kd of 2×10⁻⁵ M or lower. Preferably, said binding protein of the invention binds CD32a with a Kd of 2×10⁻⁶ M or lower, more preferably of 2×10⁻⁷ M or lower, of 2×10⁻⁸ M or lower, or most preferably of 2×10⁻⁹ M or lower, if measured by SPR. Binding proteins of the present invention binding CD32a with a Kd of 2×10⁻⁵ M or lower are shown in the Examples.

Another preferred binding protein of the present invention comprising two, three, four or more of said ankyrin repeat domains which bind CD32a will bind to CD32a with a Kd of 2×10⁻⁵ M or lower. Preferably, said binding protein of the invention binds CD32a with a Kd of 2×10⁻⁶ M or lower, more preferably of 2×10⁻⁷ M or lower, of 2×10⁻⁸ M or lower, or most preferably of 2×10⁻⁹ M or lower, if measured by SPR. Binding proteins of the present invention binding CD32a with a Kd of 2×10⁻⁵ M or lower are shown in the Examples.

The ankyrin repeat of the binding protein of the invention specifically binds CD32a. Preferably, the binding protein comprising at least one ankyrin repeat domain specifically binds human CD32a.

Preferably the binding domain of the invention is an ankyrin repeat domain or a designed ankyrin repeat domain. Examples of designed ankyrin repeat domain are shown in the examples.

In a second aspect, the invention relates to a binding protein comprising at least one ankyrin repeat domain, wherein said at least one ankyrin repeat domain specifically binds CD32a, and wherein said at least one ankyrin repeat domain comprises an amino acid sequence that has at least 70% amino acid sequence identity with one ankyrin repeat domain selected from the group consisting of SEQ ID NOs: 1-27.

As defined above, said ankyrin repeat domain binds CD32a with a Kd of 2×10⁻⁵ M or lower. Preferably, said repeat domain binds CD32a with a Kd of 2×10⁻⁶ M or lower, more preferably of 2×10⁻⁷ M or lower, of 2×10⁻⁸ M or lower, or most preferably of 2×10⁻⁹ M or lower, if measured by SPR. Binding proteins comprising an ankyrin repeat domain binding CD32a with a Kd of 2×10⁻⁵ M or lower are shown in the Examples.

A preferred binding protein of the present invention binds CD32a with a Kd of 2×10⁻⁵ M or lower. Preferably, said binding protein binds CD32a with a Kd of 2×10⁻⁶ M or lower, more preferably of 2×10⁻⁷ M or lower, of 2×10⁻⁸ M or lower, or most preferably of 2×10⁻⁹ M or lower, if measured by SPR. Binding proteins of the present invention binding CD32a with a Kd of 2×10⁻⁵ M or lower are shown in the Examples.

Preferably, the ankyrin repeat domain in a binding protein of the invention comprises an amino acid sequence with at least 70% amino acid sequence identity with randomized repeat units or randomized positions in an ankyrin repeat domain selected from the group consisting of SEQ ID NOs: 1-27. Preferably the ankyrin repeat domain in a binding protein of the invention comprises an amino acid sequence with at least 75%, more preferably at least 80%, more preferably at least 85%, more preferably at least 90%, more preferably at least 95% amino acid sequence identity. In the most preferred embodiment the ankyrin repeat domain in a binding protein of the invention comprises an amino acid sequence of one of SEQ ID NOs: 1-27.

In one embodiment a binding protein of the invention comprising at least one ankyrin repeat domain, comprises at least one ankyrin repeat module consisting of an amino acid sequence according to the following sequence motif

(SEQ ID NO: 31)
X₁DX₂X₃GX₄TPLHLAAX₅X₆GHLEIVEVLLKX₇GADVNA

X₁ to X₆ can be any amino acid except for glycine, proline or cysteine, and
X₇ can be histidine, asparagine or tyrosine, and
wherein optionally up to 5, preferably 4, 3, 2 or 1 amino acids in other than in positions denoted with X in SEQ ID NO: 31 are substituted by any amino acid. In a preferred embodiment up to 2 amino acids in other than in positions denoted with X in SEQ ID NO: 31 are substituted by any amino acid. In one embodiment of the invention in repeat module 1
   X₁ is T, E, N, S, Y, or W, preferably T,
   X₂ is E, N, T, W, H, K, N, or I, preferably E,
   X₃ is E, F, K, H, M, A, or T, preferably E,
   X₄ is L, T, A, V, F, Q, or A, preferably L,
   X₅ is L, Q, V, E, M, V, or Y, preferably L,
   X₆ is I, F, E, Q, H, V, W, or D, preferably I, and
   X₇ is Y, N, or H, preferably Y.

Thus, in a preferred embodiment of repeat module 1 X₁ is T, X₂ is E, X₃ is E, X₄ is L, X₅ is L, X₆ is I, and X₇ is Y.

In one embodiment of the invention in repeat module 2
X₁ is A, H, M, T, D, Q, S, or V, preferably A,
X₂ is M, Y, N, E, W, D, H, or T, preferably M,
X₃ is D, W, Q, S, V, F, E, or H, preferably D,
X₄ is T, R, Q, N, M, D, S, Y, or P, preferably T,
X₅ is Q, E, V, H, T, R, M, or Y, preferably Q,
X₆ is E, Y, W, F, or A, preferably E, and
X₇ is Y, or N, preferably Y,
optionally wherein the sequence has an early stop codon, optionally, wherein amino acids starting from and including position 13 are missing, preferably position 14 and most preferably position 15.

Thus, in a preferred embodiment of repeat module 2 X₁ is A, X₂ is M, X₃ is D, X₄ is T, X₅ is Q, X₆ is E, and X₇ is Y.

In one embodiment of the invention in repeat module 3
X₁ is D, A, S, Q, H, or Y, preferably D,
X₂ is F, I, H, V, Y, T, V, C, preferably F,
X₃ is W, A, F, V, E, or G, preferably W,
X₄ is H, Y, L, M, or F, preferably H,
X₅ is W, F, I, H, or R, preferably W,
X₆ is F, I, Q, D, or L, preferably F, and
X₇ is H, Y, or N, preferably H.

Thus, in a preferred embodiment of repeat module 3 X₁ is D, X₂ is F, X₃ is W, X₄ is H, X₅ is W, X₆ is F, and X₇ is H.

Thus, in a preferred embodiment repeat module 1 X₁ is T, X₂ is E, X₃ is E, X₄ is L, X₅ is L, X₆ is I, and X₇ is Y, in repeat module 2 X₁ is A, X₂ is M, X₃ is D, X₄ is T, X₅ is Q, X₆ is E, and X₇ is Y and in repeat module 3 X₁ is D, X₂ is F, X₃ is W, X₄ is H, X₅ is W, X₆ is F, and X₇ is H.

In one embodiment a binding protein of the invention comprises at least 2, or at least 3 repeat modules each comprising the repeat consensus sequence, which may be identical or different. In a preferred embodiment a binding protein of the invention comprises 3 repeat modules, which are identical or different. Preferably, the repeat domain of a binding protein of the invention comprises at least 3 repeat modules comprising the repeat consensus sequences of repeat module 1, repeat module 2 and repeat module 3.

In one embodiment of all aspects described herein, a binding protein of the invention further comprises a N-terminal and/or C-terminal capping repeat domain, preferably wherein the N-terminal and/or C-terminal capping repeat domain have an amino acid sequence different from any one ofthe ankyrin repeat modules.

Preferably the amino acid sequence of said N-terminal capping repeat domain comprises an amino acid sequence having at least 70% sequence identity to SEQ ID NO: 32, and the amino acid sequence of said C-terminal capping repeat domain comprises an amino acid sequence having at least 70% sequence identity to SEQ ID NO: 33.

Preferably, the amino acid sequence of said N-terminal capping repeat domain comprises an amino acid sequence having at least 75% more preferably at least 80%, more preferably at least 85%, more preferably at least 90%, more preferably at least 91%, 92%, 93%, 94% 95%, 96%, 97%, 98%, 99% or 100% amino acid sequence identity to SEQ ID NO: 32.

Preferably, the amino acid sequence of said C-terminal capping repeat domain comprises an amino acid sequence having at least 75% more preferably at least 80%, more preferably at least 85%, more preferably at least 90%, more preferably at least 91%, 92%, 93%, 94% 95%, 96%, 97%, 98%, 99% or 100% amino acid sequence identity to SEQ ID NO: 33.

Alternatively, the N-terminal capping repeat domain consists of the amino acid sequence according to SEQ ID NO: 32, wherein optionally up to 5, preferably up to 4, 3, 2 or 1 amino acids are substituted by any amino acid. The C-terminal capping repeat domain consists of the amino acid sequence according to SEQ ID NO: 33, wherein optionally up to 8, preferably up to 7, 6, 5, 4, 3, 2 or 1 amino acids are substituted by any amino acid.

Further preferred is a binding protein comprising any N-terminal or C-terminal capping known in the art. Examples of binding proteins of the invention comprising a N-terminal and/or C-terminal capping repeat domain are the amino acid sequences corresponding to SEQ ID NOs: 34-60 and 61-86 respectively.

In one embodiment, the binding protein further comprises another peptide or protein component, optionally in fusion with the repeat protein. In one embodiment the binding protein further comprises a lipid or lipid-like component or another non-peptide component.

In a preferred embodiment a binding protein of the invention comprising at least one ankyrin repeat domain specifically binds the extracellular domain of the CD32a receptor, preferably the Ig-like C2-type 2 domain of the CD32a ectodomain, more preferably the specifically binding comprises binding the Leucine at position 168 of the Ig-like C2-type 2 domain of CD32a. In one embodiment, said at least one ankyrin repeat domain may compete for binding to CD32a with IgG, preferably with the Fc part of IgG.

In a preferred embodiment, the CD32a is a primate CD32a, more preferably a human CD32a.

In a preferred embodiment, a binding protein of the invention discriminates between CD32a, CD32b and CD32c, preferably it does not bind CD32b or CD32c, most preferably it does not bind CD32b.

Also preferably, said binding protein does not bind CD32b indicated by a Kd above 2×10⁻⁵ M, more preferably above 2×10⁻⁴ M, or most preferably above 2×10⁻³ M for binding of CD32b.

Preferably a binding protein of the invention comprises an antigen binding protein, preferably a recombinant antigen binding protein. Preferably, said binding protein is not an antibody or fragment thereof, such as Fab or scFv fragments. Most preferably the binding protein of the invention comprises a DARPin, or is a DARPin.

In a further aspect, the invention relates to a nucleic acid comprising a nucleic acid encoding the binding proteins, the N-terminal capping repeats and/or the C-terminal capping repeats described herein.

In one embodiment, the nucleic acid sequence is operably linked to at least one transcription control unit. In one embodiment of the nucleic acid according to the invention the nucleic acid additionally comprises at least an open reading frame. In another embodiment, the nucleic acid additionally comprises at least a regulatory region of a gene. Preferably, the regulatory region is a transcriptional regulatory region, and more specifically the regulatory region is selected from the group consisting of a promoter, an enhancer, a silencer, a locus control region, and a border element. Nucleic acids according to the present invention typically comprise ribonucleic acids, including mRNA, DNA, cDNA, chromosomal DNA, extrachromosomal DNA, plasmid DNA, viral DNA or RNA, including also a recombinant viral vector. In one embodiment of the nucleic acid according to the invention the nucleic acid is DNA or RNA and in another preferred embodiment the nucleic acid is part of a plasmid or a recombinant viral vector. An inventive nucleic acid is preferably selected from any nucleic sequence encoding the amino acid sequence of the inventive binding protein. Therefore, all nucleic acid variants coding for the above mentioned inventive binding proteins with substituted amino acids including nucleic acid variants with varying nucleotide sequences due to the degeneration of the genetic code. In particular nucleotide sequences of nucleic acid variants which lead to an improved expression of the encoded fusion protein in a selected host organism, are preferred. Tables for appropriately adjusting a nucleic acid sequence to the host cell's specific transcription/translation machinery are known to a skilled person. In general, it is preferred to adapt the G/C-content of the nucleotide sequence to the specific host cell conditions. For expression in human cells an increase of the G/C content by at least 10%, more preferred at least 20%, 30%, 50%, 70% and even more preferred 90% of the maximum G/C content (coding for the respective inventive binding protein) is preferred. Preparation and purification of such nucleic acids and/or derivatives are usually carried out by standard procedures.

These sequence variants preferably lead to inventive binding proteins with binding specificity to CD32a. Therefore, inventive nucleic acid sequences code for all binding protein variants of the invention. Further, promoters or other expression control regions can be operably linked with the nucleic acid encoding the inventive binding proteins to regulate expression of the binding proteins in a quantitative or in a tissue-specific manner.

In a further aspect, the invention relates to a host cell comprising the nucleic acid described herein, which optionally expresses the binding protein. In one embodiment the cell is from an animal, preferably a vertebrate, which is preferably selected from the group consisting of a fish, a bird, or a mammal, preferably a mammal.

Further, a vector comprising, consisting essentially of, or consisting of a nucleic acid or a binding protein described herein is provided.

As already mentioned above the nucleic acid encoding the inventive binding protein may be RNA or DNA. Similarly, either the inventive nucleic acid encoding the inventive binding protein can be a linear fragment or a circularized, isolated fragment or be inserted into a vector, preferably as a plasmid or as recombinant viral DNA.

In one embodiment of all aspects, the binding protein is a molecule, optionally comprised in a particle.

In one embodiment, the particle further comprises a nucleic acid, preferably the nucleic acid is RNA or DNA. The particles may deliver the nucleic acid to cells in vitro/ex vivo as well as in vivo. Preferably the particles deliver the nucleic acid to immune cells, more preferably to CD4⁺ T cells, macrophages, monocytes, platelets, neutrophils, eosinophils, basophils and mast cells. In one embodiment, the particle is a non-viral particle. In one embodiment, the particle is a lipid based and/or polymer-based particle. In one embodiment the particle is a nanoparticle.

In one embodiment, the particle is functionalized with a binding protein or DARPin of the invention on its surface. In one embodiment, the particle is functionalized with a binding protein of the invention by linking the repeat protein to at least one particle-forming component. Functionalizing particles carrying a cargo for genetic modification of CD32a positive cells with the binders described herein results in the specific delivery of the cargo to and modification of the CD32a positive cells.

In one embodiment of all aspects described herein, the genetic modification is transient or stable. In one embodiment of all aspects described herein, the genetic modification takes place by a virus-based method, transposon-based method, or a gene editing-based method. In one embodiment, the gene editing-based method involves CRISPR-based gene editing.

Genetic modification of cells by particles described herein that are functionalized with a binding protein or a DARPin as described herein for specific targeting of CD32 positive cells, may be used ex vivo/in vitro or in vivo for delivering nucleic acid encoding antigen receptors to the cells such as CD32 positive cells to produce cells genetically modified to express the antigen receptors. Such genetic modification includes non-viral-based DNA transfection, non-viral-based RNA transfection, e.g., mRNA transfection, transposon-based systems, and viral-based systems. Non-viral-based DNA transfection has low risk of insertional mutagenesis. Transposon-based systems can integrate transgenes more efficiently than plasmids that do not contain an integrating element. Viral-based systems include the use of gamma-retroviruses and lentiviral vectors as well as AAV and adenoviral vectors. Retroviral and lentiviral vectors are enveloped particles that transduce cells permanently, while AAV and adenoviral vectors are naked particles mediating transient gene delivery at least into mitotically active cells. Display of DARPins on the surface of enveloped and naked viral vector particles is established and has been described before.

The invention also relates to a vector displaying at least one binding protein of the invention.

Nonviral vectors as for example, lipid and/or polymer-based nanoparticles may be coupled to CD32a-specific DARPins for binding to CD32a positive cells. Upon binding to CD32a positive cells, these particles are endocytosed. Their contents, for example nucleic acid encoding therapeutic genes, may be directed to the nucleus of CD32a positive cells due to, for example, the inclusion of peptides containing microtubule-associated sequences (MTAS) and nuclear localization signals (NLSs).

Therapeutic genes packaged into viral vector or nonviral vectors can e.g. be cytotoxic genes to eliminate CD32a⁺ HIV reservoir cells, or immunotherapeutic genes equipping CD32a⁺ cells with novel therapeutic activities. These are just examples of a broad variety of options that can all be combined with CD32a-targeted particles.

The inclusion of transposons flanking the nucleic acid and a separate nucleic acid, e.g., plasmid, encoding a hyperactive transposase, may allow for the efficient integration of the therapeutic nucleic acid into chromosomes.

Another possibility is to use the CRISPR/Cas9 method to deliberately place the therapeutic gene at a specific locus. For example, if the therapeutic gene was a chimeric antigen receptor (CAR), existing T cell receptors (TCRs) may be knocked out, while knocking in the CAR and placing it under the dynamic regulatory control of the endogenous promoter that would otherwise moderate TCR expression.

Accordingly, besides nucleic acid encoding an antigen receptor the particles described herein may also deliver as cargo gene editing tools like CRISPR/Cas9 (or related) or transposon systems like sleeping beauty or piggy bag. Such tools (e.g. transposase, gene editing tools like CRISPR/Cas9) for genomic integration/editing may be delivered as protein or coding nucleic acid (DNA or RNA). Nevertheless, also delivery of mRNA is an option to induce transient expression of therapeutic genes.

A particular example with respect to CD32a⁺, chronically infected HIV-positive cells is the delivery of CRISPR/Cas tools to excise and/or inactivate the integrated proviral HIV genome. Delivery of these editing machineries precisely to the chronically infected HIV reservoir cells can be obtained with any of the vector systems described above when they display the CD32a-specific DARPin on their surface.

In one embodiment of all aspects of the invention, the cells genetically modified to express a therapeutic gene are stably or transiently transfected with nucleic acid encoding the therapeutic gene. Thus, the nucleic acid encoding the therapeutic gene is integrated or not integrated into the genome of the cells.

In one embodiment of all aspects of the invention, the cells described herein may be autologous, allogeneic or syngeneic to the subject to be treated. In one embodiment, the present disclosure envisions the removal of cells from a patient and the subsequent re-delivery of the cells to the patient. In one embodiment, the present disclosure does not envision the removal of cells from a patient. In the latter case all steps of genetic modification of cells are performed in vivo.

In a further aspect, the invention relates to a composition comprising the binding protein described herein, the nucleic acid described herein, the host cell described herein, the particle described herein, or a plurality thereof.

In a further aspect, the invention relates to pharmaceutical composition comprising at least one of the binding protein, the nucleic acid, the host cell, the vector, and/or the particle described herein.

The pharmaceutical composition may further comprise one or more carriers and/or excipients, all of which are preferably pharmaceutically acceptable. According to the invention, a pharmaceutical composition contains an effective amount of the active agents, e.g., the polypeptide, nucleic acid, vector, or cell described herein, to generate the desired reaction or the desired effect. A pharmaceutical composition in accordance with the present invention is preferably sterile. Pharmaceutical compositions can be provided in a uniform dosage form and may be prepared in a manner known per se. A pharmaceutical composition in accordance with the present invention may, e.g., be in the form of a solution or suspension.

Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. The pharmaceutical compositions of the present invention may be administered orally, parenterally, by inhalation spray, topically, rectally, nasally, buccally, vaginally or via an implanted reservoir. The term parenteral as used herein includes subcutaneous, intravenous, intramuscular, intra-articular, intra-synovial, intrasternal, intrathecal, intrahepatic, intralesional and intracranial injection or infusion techniques. Preferably, the pharmaceutical compositions are administered orally, intraperitoneally or intravenously. Sterile injectable forms of the pharmaceutical compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

The inventive pharmaceutical composition is preferably suitable for the use in treating, preventing, or diagnosing a condition. Preferably the condition is associated with altered CD32a expression levels in a subject. Altered CD32a expression levels refers to and increased or decreased expression of CD32a as compared to a control level.

In one aspect of the invention, the herein disclosed binding proteins, nucleic acids, vectors, particles, host cells, compositions or pharmaceutical compositions are used as a medicament. Preferably the medicament is an immunomodulatory agent, preferably in the context of allergy, autoimmune diseases, or immunodeficiency disorders such as those caused by human immunodeficiency viruses (HIV).

The herein disclosed binding proteins, nucleic acids, vectors, particles, host cells, compositions or pharmaceutical compositions can be used in treating, preventing or diagnosing a condition. Preferably the condition is associated with CD32a expression levels, preferably associated with altered CD32a expression levels and/or wherein targeting of CD32a is desired.

In a further aspect, the invention relates to a kit comprising herein disclosed binding proteins nucleic acids, host cell, vectors, particles, composition or pharmaceutical compositions. Preferably the kit is for use in the treatment, prevention or diagnosis of a condition. Preferably the kit further comprises instructions for using the kit in a method of treating, preventing or diagnosing the condition. Preferably said condition is a condition associated with CD32a expression levels, preferably associated with altered CD32a expression levels, and/or wherein targeting of CD32a is desired.

In an embodiment of all aspects described herein, the condition associated with altered CD32a expression levels is an allergy, an autoimmune disease/an immune deficiency, or a heparin-induced thrombocytopenia. The immune deficiency includes HIV, rheumatoid arthritis, Huntington disease, α-anti-Trypsin deficiency, as well as cancer selected from colon cancer, melanomas, kidney cancer, lymphoma, acute myeloid leukemia (AML), acute lymphoid leukemia (ALL), chronic myeloid leukemia (CML), chronic lymphocytic leukemia (CLL), gastrointestinal tumors, lung cancer, gliomas, thyroid cancer, mamma carcinomas, prostate tumors, hepatomas, diverse virus-induced tumors such as e.g. papilloma virus induced carcinomas (e.g. cervix carcinoma), adeno carcinomas, herpes virus induced tumors (e.g. Burkitt's lymphoma, EBV induced B cell lymphoma), Hepatitis B induced tumors (Hepato cell carcinomas), HTLV-1 und HTLV-2 induced lymphoma, akustikus neurinoma, lung cancer, pharyngeal cancer, anal carcinoma, glioblastoma, lymphoma, rectum carcinoma, astrocytoma, brain tumors, stomach cancer, retinoblastoma, basalioma, brain metastases, medullo blastoma, vaginal cancer, pancreatic cancer, testis cancer, melanoma, bladder cancer, Hodgkin syndrome, meningeoma, Schneeberger's disease, bronchial carcinoma, pituitary cancer, mycosis fungoides, gullet cancer, breast cancer, neurinoma, spinalioma, Burkitt's lymphoma, lyryngeal cancer, thymoma, corpus carcinoma, bone cancer, non-Hodgkin lymphoma, urethra cancer, CUP-syndrome, oligodendroglioma, vulva cancer, intestinal cancer, oesphagus carcinoma, small intestine tumors, craniopharyngeoma, ovarial carcinoma, ovarian cancer, liver cancer, leukemia, or cancers of the skin or the eye; etc. Preferably the condition is HIV or rheumatoid arthritis, more preferably HIV.

There are a variety of alternative techniques and procedures available to those of skill in the art which would similarly permit one to successfully practice the intended invention.

### EXAMPLES

### Example 1: CD32a-DARPin Identification

### Ribosome Display

Ribosome display selection was carried out as described by Hartmann and colleagues (Hartmann et al. 2018). In total, up to five selection rounds were carried out. As bait protein the extracellular domain of CD32a tagged with a C-terminal His-tag (CD32a-His, Sino-Biologicals) with or without biotinylation was used. Counter selection was performed for CD32b using recombinant CD32b protein (CD32b-His, Sino-Biologicals). The first three selection rounds were performed with immobilized, biotinylated CD32a-His (Sino Biologicals, 10374-H27H1-B), including pre-selection steps against neutravidin or streptavidin to exclude selection of sticky DARPins. The fourth and fifth selection round was carried out in solution. In the fourth selection round an off-rate selection with unbiotinylated CD32a-His protein (Sino Biologicals, 10374-H27H) was performed and the fifth selection round included a counter selection with unbiotinylated CD32b-His (Sino Biologicals, 10259-H27H) protein to select binders with high affinity and specificity for CD32a.

Briefly, for the first three selection rounds, the translated VV-N2C and VV-N3C DARPin libraries were subjected to pre-panning steps with immobilized neutravidin or streptavidin (both 66 nM). For on-target selection, the libraries were incubated with immobilized, biotinylated CD32a-His (425.5 nM). The resulting DARPin libraries were amplified and used as template for the next selection round. After three rounds with immobilized target protein, the fourth and fifth round of selection was carried out with target in solution. In the fourth round the libraries were first exposed to the biotinylated CD32a-His target (17 pmol) and afterwards exposed as an off-rate selection to 10-fold excess unbiotinylated CD32a (170 pmol). Finally, the fifth round was divided into two approaches: (i) In one approach the libraries were pre-selected with unbiotinylated CD32b-His protein (17 pmol) before they were exposed to the biotinylated CD32a-His protein (17 pmol), (ii) in the other approach the libraries were first exposed to the biotinylated CD32a-His protein and afterwards counter-selected with unbiotinylated CD32b-His protein (170 pmol). After the fifth selection round, DARPin-encoding DNA fragments were cloned into E. coli and analyzed for CD32a binding by single clone analysis.

### DARPin Expression and Crude Lysate Preparation

To test selected DARPins for specific binding to CD32a after the fifth selection round, DNA fragments were cloned into the bacterial expression vector pQE-HisHA and transformed into E. coli XL 1-blue as described before (Hartmann et al. 2018). Single clones were picked and cultured overnight in 600 μl 2YT medium (2YT, 1% glucose, 100 g/ml ampicillin) at 37° C. before cultures were diluted in 1 ml 2YT medium to an OD₆₀₀ of 0.1 and expression of the DARPins was induced after 1 h cultivation by addition of 100 μL of 5.5 mM isopropyl-b-D-thiogalactopyranosid (IPTG) in 2YT medium. After culture for 5 h at 37° C., bacteria were harvested by centrifugation and pellets were stored overnight at −80° C. The next day, pellets were thawed on ice and lysed by addition of B-PERII solution (Thermo Scientific) and subsequent incubation at room temperature for 2 hours. The lysed pellet was vortexed and diluted 1:10 with TBS before cell debris removal by centrifugation. The supernatant containing crude DARPin was aliquoted and stored at −80° C. until use in cellular binding assays. Crude lysate preparations were always handled on ice and subjected to a maximum of three freeze-thaw cycles to avoid loss of protein quality. In total 285 DARPin clones from the fifth selection round were expressed in E. coli and tested for binding to cell surface expressed CD32a. From the tested DARPin clones, promising candidates were identified and sequenced using standard sequencing technologies to obtain DNA and protein sequences.

### Cell-Based Binding Assays

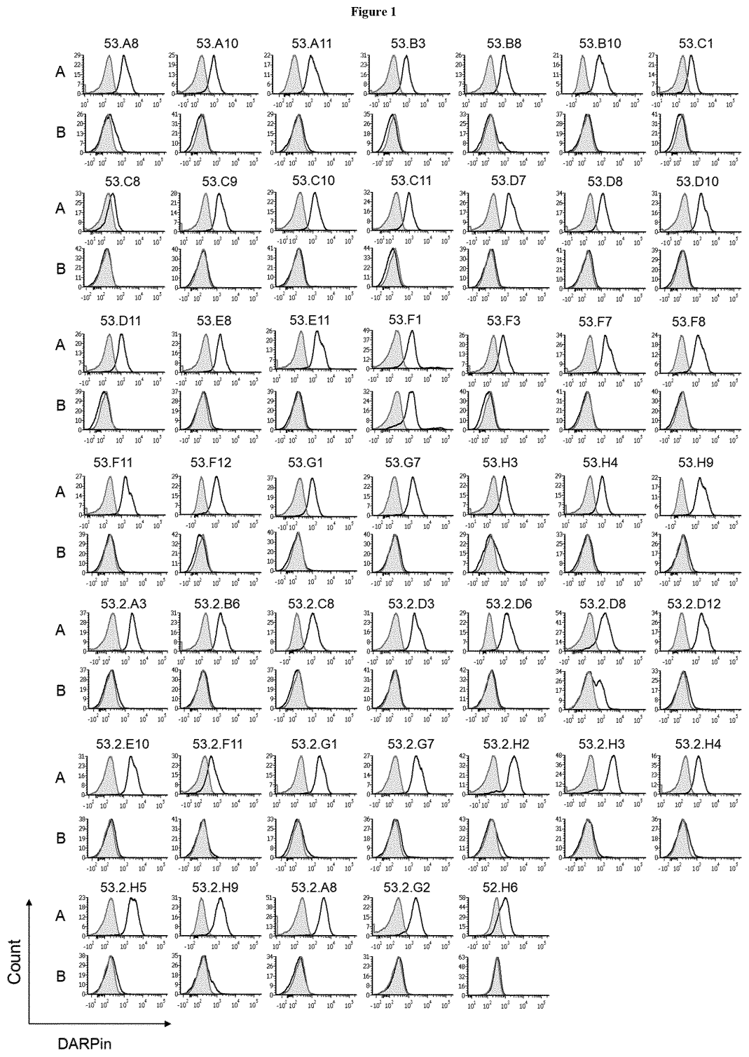

To analyze the CD32a-binding ability of the selected DARPin clones, a cell-based binding assay was carried out using stable CD32a or CD32b receptor expressing cell lines which were generated by lentiviral transduction and verified for proper cell surface expression by CD32 antibody staining (data not shown). In brief, 8×10³-1×10⁵ cells were incubated with 5-25 μl of crude DARPin extracts for 60 min at 4° C. Following incubation, cells were washed twice, stained with fluorescently labelled antibodies detecting the DARPin and analyzed by flow cytometry. Of the 285 clones tested, 45 individual DAR-Pin clones specifically bound to human CD32a-positive cells, while only two DAR-Pins (53.F11, 53.2.D38) showed off-target binding to CD32b-positive cells (FIG. 1). Notably, beside one DARPin (52.H6) all other identified CD32a binding DARPins were derived from the VV-N3C library.

TABLE 1
Sequences of the identified CD32a-specific DARPins

| SEQ | | |
|---|---|---|
| ID | | |
| NO: | CLONE | SEQUENCE |
| 1 | 53.H3 | DLGKKLLEAARAGQDDEVRILMANGADVNANDTKGATPLHLAAVEGHLEIVEVLLKHGADVNAMDN |
| | | QGQTPLHLAAQWGHLEIVEVLLKYGADVNAADIAGYTPLHLAAWFGHLEIVEVLLKYGVDVNAQDKF |
| | | GKTPFDLAIDNGNEDIAEVLQKAA |
| 2 | 53.2.F11 | DLGKKLLEAARAGQDDEVRILMANGADVNASDWHGATPLHLAAEQGHLEIVEVLLKHGADVNATDE |
| | | SGNTPLHLAAVFGHLEIVEVLLKNGADVNASDHFGLTPLHLAAWFGHLEIVEVLLKHGADVNAQDKF |
| | | GKTPFDLAIDNGNEDIAEVLQKAA |
| 3 | 53.C8 | DLGKKLLEAARAGQDDEVRILMANGADVNATDKAGFTPLHLAAYVGHLEIVEVLLKNGADVNAHDH |
| | | QGDTSLHLAAT |
| 4 | 53.D8 | DLGKKLLEATRAGQDDEVRILMANGADVNATDEEGLTPLHLAALIGHLEIVEVLLKYGADVNAADMD |
| | | GTTPLHLAAQEGHLEIVEVLLKYGADVNADDFWGHTPPHLAAWFGHLEIVEVLLKHGADVNAQDKF |
| | | GKTPFDLAIDNGNEDIAEVLQKAA |
| 5 | 53.2.A8 | DLGKKLLEATRAGQDDEVRILMANGADVNATDEEGLTPLHLAALIGHLEIVEVLLKYGADVNAADMD |
| | | GTTPLHLAAQEGHLEIVEVLLKYGADVNADDFWGHTPLHLAAWFGHLEIVEVLLKHGADVDAQDKF |
| | | GKTPFDLAIDNGNEDIAEVLQKAA |
| 6 | 53.2.D6 | DLGKKLLDAASAGQDDEVRILMANGADVNATDEEGLTPLHLAALIGHLEIVEVLLKYGADVNAADMD |
| | | GTTPLHLAAQEGHLEIVEVLLKYGADVNADDFWGHTPLHLAAWFGHLEIVEVLLKHGADVNAQDKF |
| | | GKTPFDLAIDNGNEDIAEVLQKAA |
| 7 | 53.2.G2 | DLGKKLLEAARAGQDDEVRILMANGADVNATDEEGLTPLHLAALIGHLEIVEVLLKYGADVNAADMD |
| | | GTTPLHLAAQEGHLEIVEVLLKYGADVNADDFWGHTPLHLAAWFGHLEIAEVLLKHGTDVNAQDKFG |
| | | KTPFDLAIDNGNEDIAEVLQKAA |
| 8 | 53.A10 | DLGKKLLEAARAGQDDEVRILMSNGADVNAWDKEGVTPLHLAAVHGHLEIVEVLLKHGADVNAQDD |
| | | VGMTPLHLAAHWGHLEIVEVLLKYGADVNAQDYAGMTPLHLAAFQGHLEIVEVLLKHGADVNAQDK |
| | | FGKTPFDLAIDNGNEDIAQVLQKAA |
| 9 | 53.C1 | DLGKKLLEAARVGQDDEVRILMANGADVNATDNEGQTPLHLAAMWGHLEIVEVQLKHGADVNASDT |
| | | FGSTPLHLAARFGHLEIVEVLLKYGADVNAADTEGFTPLHLAAIDGHLEIVEVLLKHGADVNAQDKFG |
| | | KTPFDLAIDNGNEDIAEVLQKAA |
| 10 | 53.D11 | DLGKKLLEAARAGQDDEVRILMANGADVNATDITGATPLHLAAMWGHLEIVEVLLKYGADVNAQDYE |
| | | GYTTLHLAAMWGHLEIVEVLLKYGADVNAHDVAGHTPLHLATHFGHLEIVEVLLKHGADVNAQDKFG |
| | | KTPFDLAIDNGNEDIAEVLQKAA |
| 11 | 53.G1 | DLGKKLLEATRAGQDDEVRILMSNGADVNAWDKEGVTPLHLAAVHGHLEIVEVLLKHGADVNAQDD |
| | | VGMTPLHLAAHWGHLEIVEVLLKYGADVNAQDYAGMTPLHLAAFQGHLEIVEVLLKHGADVNAQDK |
| | | FGKTPFDLAIDNGNEDIAEVLQKAA |
| 12 | 53.2.C8 | DLGKKLLEAARAGQDDAVRILMANGADANAYDHMGETPLHLAAMEGHLEIVEVLLKHGADVNADDW |
| | | DGQTPLHLAAHWGHLEIVEVLLKYGADVKEQDVVGYTPLHLAAWIGHLEIVEVLLKNGADVNAQDKF |
| | | GKTPFDLAIDNGNEDIAEVLQKAA |
| 13 | 53.A8 | DLGKKLLEATRAGQDDEVRILMANGANVNATDEEGLTPLHLAALDGHLEIVEVLLKYGADVNADDMH |
| | | GPTPLHLAAYAGHLEIVEVLLKYGADVNAYDCGGHTPLHLAARLGHLEIVEVLLKYGADVNAQDKFG |
| | | KTPFDLAIDNGNEDIAEVLQKAA |
| 14 | 53.G7 | DLGKKLLEATRAGQDDEVRILMANGADVNATDEEGLTPLHLAVLIGHLEIVDVLLKYGADVNAADMD |
| | | GTTPLHLAAQEGHLEIVEVLLKYGADVNADDFWGHTPLHLAAWFGHLEIVEVLLKHGADVNAQDKF |
| | | GKTPFDLAIDNGNEDIAEVLQKAA |
| 15 | 53.H9 | DLGKKLLEATRAGQDDEVRILMANGADVNATDEEGLTPLHLAALIGHLEIVEVLLKYGADVNAVDMD |
| | | GTTPLHLAAQEGHLEIVEVLLKYGADVNADDFWGHTPLHLAAWFGHLEIVEVLLKHGADVNAQDKF |
| | | GKTPFDLAIDNGNEDIAQVLQKAA |
| 16 | 53.B8 | DLGKKLLEATRAGQDDEVRILMTNGTDVNATDEEGLTPLHLAALIGHPEIVEVLLKYGADVNAADMD |
| | | GTTPLHLAAQEGHLEIVEVLLKYCADVNADDFWGHTPLHLAAWFGHLEIVEVLLKYGADVNAQDKFG |
| | | KTPFDLAIDNGNEDIAEVLQKAA |
| 17 | 53.B10 | DLGKKLLEATRAGQDDEVRILMANGADVNATDEEGLTPLHLAALIGHLEIVEVLLKYGADVNAADMD |
| | | GTTPLHLAAQEGHLEIVEVLLKYGADVNADDFWGHTPLHLAAWFGHLEIVEVLLKNGADVNAQDKF |
| | | GKTPFDLAIDNGNEDIAEVLQKAA |
| 18 | 53.2.H4 | DLGKKLLEATRAGQDDEVRILMANGADVNTTDEEGLTPLHLAALIGHLEIVEVLLKYGADVNAADMD |
| | | GTTPLHLAAQEGHLEIVEVLLKYGADVNADDFWGHTPLHLAAWFGHLEIVDVLLANVADVNANSAQS |
| | | KTPFDLAIDNGNEDIAEVLQKAA |
| 19 | 53.2.H9 | DLGKKLLEATRAGQDDEVRILMANGADVNATDEEGLTPLHLAALIGHLEIVEVLLKYGADVNAADMD |
| | | GTTPLHLAVQEGHLEIVEVLLKYGADVNADDFWGHTPLHLAAWFGHLEIVEVLLKYGADVNAQDKFG |
| | | KTPFDLAIDNGNEDIAEALQKAA |
| 20 | 53.D7 | DLGKKLLEAARAGQDDEVRILMANGADVNATDEEGLTPLHLAALIGHLEIVEVLLKYGADVNAADMD |
| | | GTTPLHLAAQEGHLEIVEVLLKYGADVNADDFWGHTPLHLAAWFGHLEIVEVLLKNGADVNAQDKF |
| | | GKTPFDLAIDNGNEDIAEVLQKAA |
| 21 | 53.E11 | DLGKKLLEAARAGQDDEVRILMANGADVNATDEEGLTPLHLAALIGHLEIVEVLLKYGTDVNAADMD |
| | | GTTPLHLAAQEGHLEIVEVLLKYGADVNADDFWGHTPLHLAAWFGHLEIVEVLLKHGADVNAQDKF |
| | | GKTPFDLAIDNGNEDIAEVLQKAA |
| 22 | 53.2.B6 | DLGKKLLEATRAGQDDEVRILMANGADVNATDEEGLTPLHLAALIGHLEIVEVLLKYGADVNAADMD |
| | | GTTPLHLAAQEGHQEIVEVLLKYGADVNADDFWGHTPLHLAAWFGHLEIVEVLLKHGADVNAQDKF |
| | | GKIPFDLAIDNGNEDIAEVLQKAA |
| 23 | 53.2.E10 | DLGKKLLEATRAGQDDEVRILMANGADVNTTDEEGLTPLHLAALIGHLEIVEVLLKYGADVNAADMD |
| | | GTTPLHLAAQEGHLEIVEVLLKYGADVNADDFWGHTPLHLAAWFGHLEIVEVLLKHGADVNAQDKF |
| | | GKTPFDLAIDNGNEDIAEVLQKAA |
| 24 | 53.2.H5 | DLGKKLLEATRAGQDDEVRILMANGADVNATDEEGLTPLHLAALIGHLEIVEVLLKYDADVNAADMD |
| | | GTTPLHLAAQEGHLEIVEVLLKYGADVNADDFWGHTPLHLAPWFGHLEIVEVLLNHGADVNAQDKF |
| | | GKTPFDLAIDNGNEDLAEVLQKAA |
| 25 | 53.F11; | DLGKKLLEATRAGQDDEVRILMANGADVNATDEEGLTPLHLAALIGHLEIVEVLLKYGADVNAADMD |
| | 53.D10 | GTTPLHLAAQEGHLEIVEVLLKYGADVNADDFWGHTPLHLAAWFGHLEIVEVLLKHGADVNAQDKF |
| | | GKTPFDLAIDNGNEDIAQVLQKAA |
| 26 | 53.A11; | DLGKKLLEATRAGQDDEVRILMANGADVNATDEEGLTPLHLAALIGHLEIVEVLLKYGADVNAADMD |
| | 53.C9; | GTTPLHLAAQEGHLEIVEVLLKYGADVNADDFWGHTPLHLAAWFGHLEIVEVLLKHGADVNAQDKF |
| | 53.F7: | GKTPFDLAIDNGNEDIAEVLQKAA |
| | 53.F8; | |
| | 53.2.H3; | |
| | 53.2.D3; | |
| | 53.2.H2 | |
| 27 | 52.H6 | DLGKKLLEAARAGQDDEVRILIANGADVNAEDNFGTTPLHLAAQFGHLEIVEVLLKNGVDVNAHDYW |
| | | GRTPLYLAAEYGHLEIVEVLLKYGADVNAQDKFGKTPFDLAIDNGNEDIAEMLQKAAAA |

### DARPin Sequence Analysis

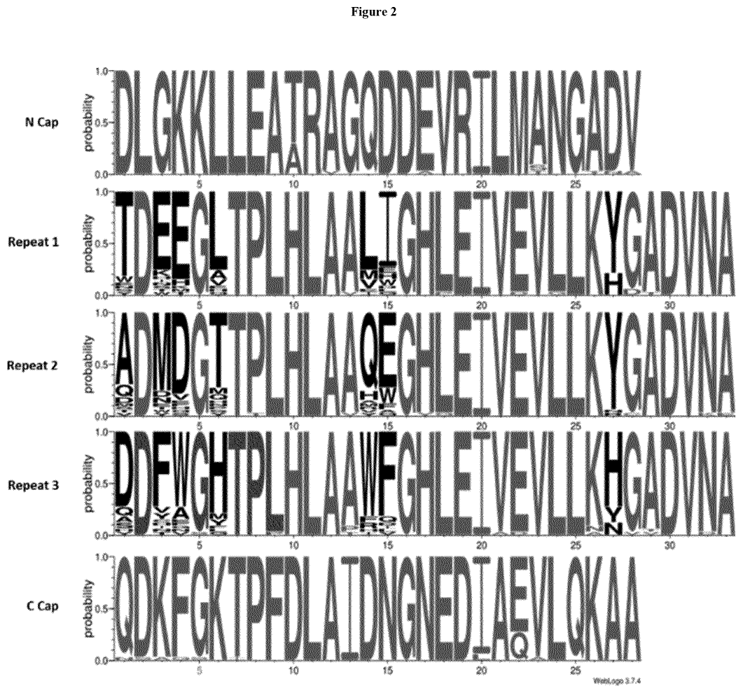

Sequencing revealed that 34 out of 45 identified CD32a-specific candidates resemble the common DARPin architecture of which 27 had a unique amino acid sequence (Table 1). For the remaining DARPin candidates the typical DARPin architecture was not complete (early sequence truncation, etc.), so that these candidates were not further considered. Each individual DARPin clone comprises at least one diversified repeat domain flanged by a N- and C-terminal capping repeat domain. Each repeat unit comprises the same structural motif wherein more than 69.8% of the framework residues of the 33 N3C DARPins are homologous to each other. The diversified amino acids at position 1, 3, 4, 6, 14 and 15 in each diversified repeat domain can contain any amino acids except glycine, proline or cysteine. The diversified amino acid position 27 in each diversified repeat domain can contain a histidine, asparagine or tyrosine. Evaluation of the diversified position among the individual DAR-Pin clones and repeat motifs revealed that the selected CD32a-DARPins preferentially harbor a threonine (T) at position 1, a glutamate (E) at position 3 and 4, a leucine (L) at position 6 and 14, a isoleucine (1) at position 15 and a tyrosine (Y) at position 27 in the first diversified repeat, a alanine (A) at position 1, a methionine (M) at position 3, a aspartate (D) at position 4, a threonine (T) at position 6, a glutamine (Q) at position 14, a glutamate (E) at position 15 and a tyrosine (Y) at position 27 in the second diversified repeat and a aspartate (D) at position 1, a phenylalanine (F) at position 3 and 15, a tryptophan (W) at position 4 and 14, a histidine (H) at position 6 and 27 in the third diversified repeat (FIG. 2 and Table 1).

### Example 2: Specific Labelling of CD32a-Positive Cell Populations with Purified CD32a-DARPins

To identify CD32a-specific DARPins able to discriminate CD32a and CD32b expressing cell populations, 6 out of 34 DARPin candidates were investigated in more detail using purified CD32a-DARPins as described by Hartmann and colleagues (Hartmann et al. 2018) with slight modifications. In brief, for each DARPin a single colony was inoculated in lysogeny broth (LB) complete media at 30° C. overnight. On the next day 100 ml of fresh LB complete were inoculated with 5 ml of the overnight culture. At an optical density OD of 0.7 the protein expression was induced by addition of 300 M IPTG. After cultivation for 4 h at 37° C., bacteria were harvested by centrifugation and pellets were stored at −80° C. Pellets were thawed on ice and either re-suspended in lysis buffer and disrupted by sonification for mechanical extraction or mixed with B-PERII for a chemical extraction. Cell debris were removed by centrifugation, the supernatant re-stocked and glycerol-buffer (2.5×) was added. The purification of the DARPin on Ni-nitrilotriacetic acid (NTA) Agarose columns was performed according to the manufacturer. During the procedure, samples were taken at different times for SDS PAGE. The elution fraction was dialyzed against PBS over night at 4° C. using a dialysis tubing (MWCO depends on used extraction method). The dialyzed DARPin protein was measured by Bradford Assay and if necessary supplemented with glycerol and protease inhibitor for storage.

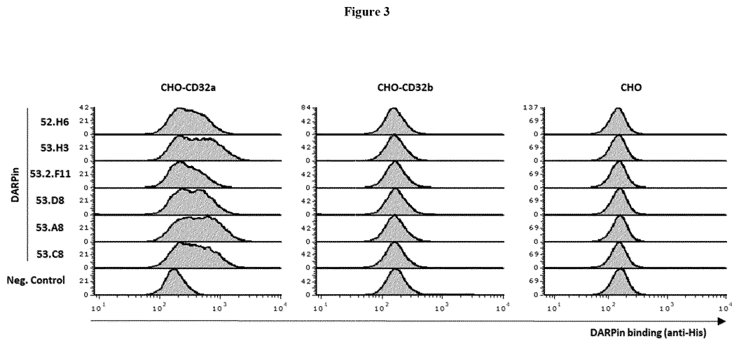

To analyze the CD32a-binding ability of the selected purified DARPin proteins, a cell-based binding assays were carried out using either transient or stable CD32a or CD32b expressing cell lines. In brief, 8×10⁴-1×10⁵ cells were incubated with different amounts of purified DARPins. Following incubation, cells were washed, stained with fluorescently labelled antibodies detecting the His tag of the DARPin and were analyzed by flow cytometry. The selected purified DARPins were able to distinguish between CD32+ and off-target cells with variable cell staining intensity across the DARPin candidates (FIG. 3). Binding of available CD32a cell surface receptors seems to be accomplished with a DARPin concentration of 1 μg or below.

### Example 3: Mapping of Binding Site of CD32a-DARPins

The sequence of the extracellular domains of CD32a and CD32b differ only by 12 amino acids, whereby the first both amino acid are not considered in this study because of their proximity to the signal peptide (FIG. 4). To identify which of those amino acids contribute to CD32a DARPin binding, chimeric CD32b proteins harboring 1 to 5 CD32a specific amino acids in their extracellular domains (Table 2) were synthesized, expressed on SupT1 cells and used as target population in a cell based binding. assay as described above. All chimeric CD32b proteins were well expressed on the cell surface were able to bind IgG-Fc to various extent (data not shown). Interestingly, only chimeric CD32b variants which harbor a leucine instead of a serine at position 135, counted from the start of the ectodomain, were able to mediate CD32a-DARPin binding (FIG. 5). That this single amino acid exchange in the ECD of CD32b is sufficient to enable CD32a-DARPin binding and leads to the discrimination of CD32a and CD32b by the selected DARPins was further confirmed by binding analysis of the chimeric CD32b.mut10 receptor carrying only the single point mutation S135L. In contrast, the single point mutation N138T in the chimeric CD32b.mut11, which is present also in the chimeric CD32b.mut4, CD32b.mut6, CD32b.mut8 and CD32b.mut9 versions did not lead to DARPin binding. Notably, this leucine is placed in the Ig-like C2-type 2 domain of CD32a which is the binding region for the Fc part of IgG (Maxwell et al, 1999; Ramsland et al, 2011), the native ligand of CD32a receptor.

To analyse whether CD32a-DARPins compete with IgG antibodies for CD32a binding a competition assay was performed. Prior addition of IgG antibodies to the cells showed reduced to non-existent binding of the DARPin to CD32a+ cells (data not shown). Thus, it can be assumed that there is a positive correlation between the IgG binding region on the CD32a receptor surface and the DARPin binding region.

TABLE 2
Overview of mutations and their position in the ectodomain of CD32b.
Exchange of serine to leucine at position 135 is depicted in bold.

| | Position in the | Location in the |
|---|---|---|
| Declaration | Ectodomain | Ectodomain |
| CD32b.mut1 | Q15P | Ig-like C2-type 1 domain |
| CD32b.mut2 | R30Q, T32A, H33R | Ig-like C2-type 1 domain |
| CD32b.mut3 | K133Q | Ig-like C2-type 2 domain |
| CD32b.mut4 | S135L, N138T | Ig-like C2-type 2 domain |
| CD32b.mut5 | Q15P, R30Q, T32A, H33R | Ig-like C2-type 1 domain |
| CD32b.mut6 | K133Q, S135L, N138T | Ig-like C2-type 2 domain |
| CD32b.mut7 | K133Q, Y163F | Ig-like C2-type 2 domain |
| CD32b.mut8 | V107M, S135L, N138T | Ig-like C2-type 2 domain |
| CD32b.mut9 | V107M, K133Q, S135L, | Ig-like C2-type 2 domain |
| | N138T, Y163F | |
| CD32b.mut10 | S135L | Ig-like C2-type 2 domain |
| CD32b.mut11 | N138T | Ig-like C2-type 2 domain |

### Example 4: CD32a-Specific Gene Transfer by LVs Displaying CD32a-DARPins

DARPins were previously shown to be a suitable tool to retarget lentiviral vectors to a receptor of choice (Hartmann et al, 2018; Frank et al, 2018). For this purpose, the DARPins have to be genetically fused to the glycoprotein of the Nipah virus (NiV-G). 7 identified DARPin candidates (53.2.G2, 53.2A8, 53C8, 52H6, 53.2.F11, 53D8, 53H3) were cloned to the C-terminus of N-terminally truncated and natural receptor blinded NiV-G as described by Frank et al, 2020. To analyze whether the binding ability and specificity of the selected CD32a-DARPins is not abolished by fusion to the NiV-G protein and display on 293T cells, a cell-based binding assay was carried out with 293T cells transiently expressing the various DARPin-NiV-G proteins.

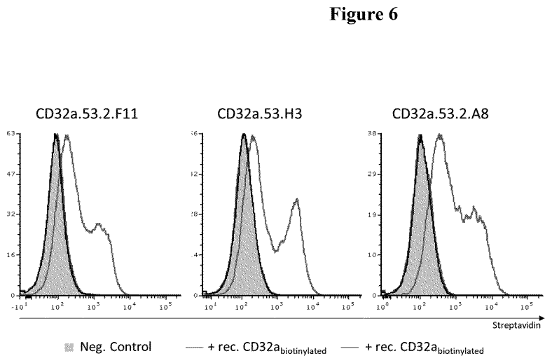

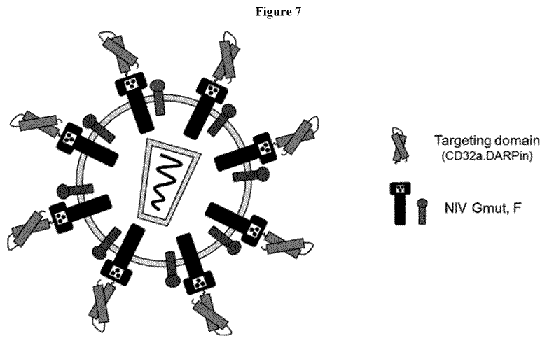

In brief, DARPin presenting cells were generated by transient transfection of HEK293T cells using PEI. For the transfection mix 400 ng of total DNA was mix with 30 μl DMEM without additives and added to 30 μl DMEM supplemented with 2 μl PEI solution per 24-well plate. After incubation for 15 min at room temperature, the transfection mix was added to the cells 6 hours later, the medium was replaced by fresh cell culture medium. At day 2 post transfection 1×10⁵ cells were incubated with 250 ng biotinylated recombinant CD32a or CD32b protein. Following incubation, cells were washed, stained with fluorescently labelled streptavidin and analyzed by flow cytometry. FIG. 7 shows CD32a-DARPin fusion to NiV-G and expression in mammalian cells retains their CD32a receptor binding capability as well as receptor specificity. These findings are consistent with previous results (FIG. 1, FIG. 3 & FIG. 6) showing that the selected DARPins have a high specificity for CD32a.

In a next step, receptor targeted lentiviral vectors displaying those DARPin candidates in their envelope and harboring GFP as a reporter (CD32a-LV) were screened for their ability to mediate CD32a-specific transgene delivery as described before (Frank et al., 2020). A schematic composition of the CD32a-LV is shown in FIG. 7.

In brief, LV vector particles were generated by transient transfection of HEK293T cells using PEI either in a small scale format (12-well, total of 0.8 g DNA) or large scale format (T175 flask, total of 35 g DNA). For the transfection mix of a small scale production, 800 ng of total DNA was mix with 53 μl DMEM without additives and added to 50 μl DMEM supplemented with 3 μl PEI solution per 12-well plate. After incubation for 15 min at room temperature, the transfection mix was added to the cells. 6 hours later, the medium was replaced by fresh cell culture medium. At day 2 post transfection, cell supernatant containing the vector particles were collected and centrifuged to remove cell debris for a small scale production or filtered. The supernatant was used for transduction experiments. LV production in a large scale is described by Frank et al, 2020.

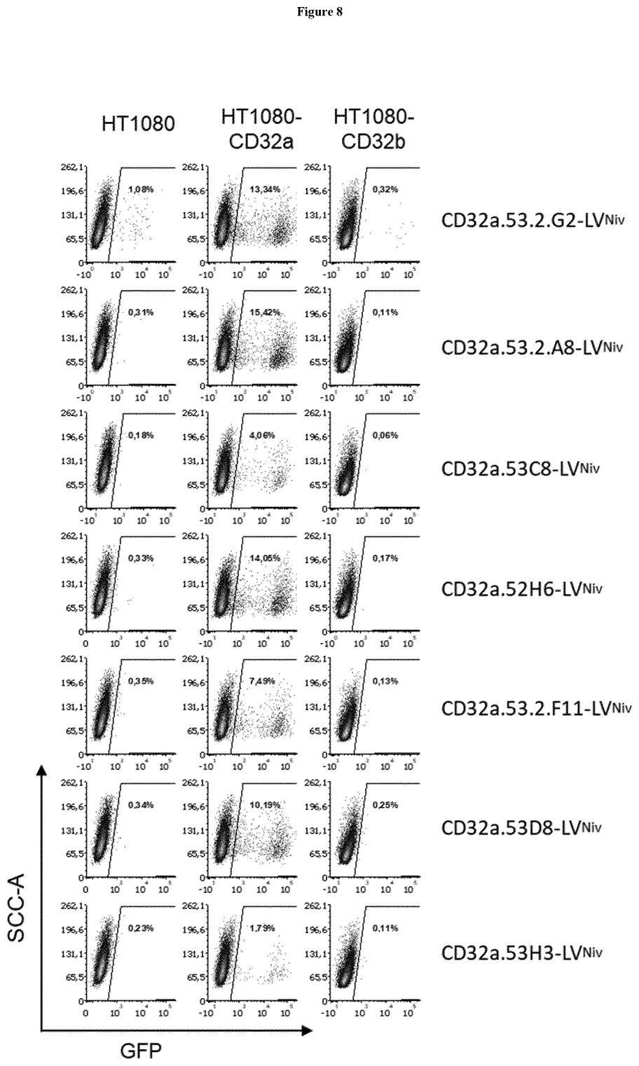

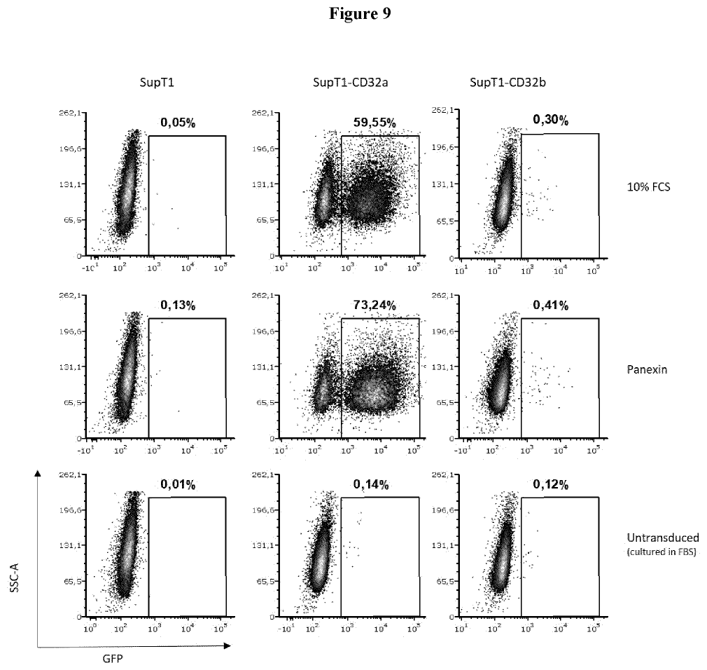

Generated CD32a-LV particles were screened for their ability to transduce CD32a-positive (HT1080-CD32a) and negative (HT1080-wt, HT1080-CD32b) cell lines. All 7 tested candidates mediated CD32a-specific gene transfer upon display on LV particles (FIG. 8). From those DARPin 53.2.F11 and 53.H3 were produced in a large scale production (20× T175 culture flasks) and further evaluated for their transduction efficiency on SupT1 cells with and without CD32a or CD32b receptor expression (FIG. 9; data not shown for CD32a.53H3-Lentiviral vector). Interestingly, the usage of serum-free medium (Panexin) leads to a higher transduction of the target cells. This finding might be explained by the presence of IgG in fetal bovine serum (FCS) which might act as a competitor to the CD32a-DARPins.

### Example 5: Kinetic Measurement of Selected CD32a-DARPins

To determine the binding affinity of the selected DARPins to human CD32a surface plasma resonance (SPR) measurements were carried out on a Biacore T200 instrument (Cytiva). To further proof the specificity of the DARPin potential binding to human CD32b was analyzed as well.

In brief, a neutravidin NA chip (Cytiva) was prepared according to the manufacturer's protocol and immobilized with 110 RU human biotinylated CD32a (or 985 RU) and 114 RU human biotinylated CD32b protein, respectively. As running buffer 20 mM HEPES, 150 mM NaCl and 0.05% Tween 20 was used. DARPin samples were injected in varying concentrations for 3 min followed by a 15 min dissociation phase before regeneration with 10 mM NaOH was performed. The binding kinetic of the protein-protein interaction was analyzed and fitted using Biacore T200 evaluation software. The signal of an uncoated reference flow cell was subtracted from each measurement.

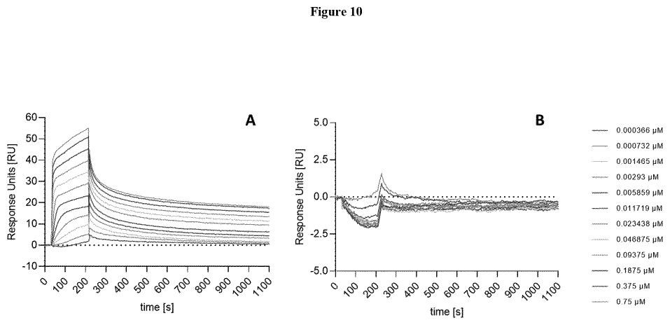

In SPR experiments, the selected DARPins were specific to recombinant CD32a protein and did not cross-react with CD32b protein or neutravidin. We determined the binding kinetic of DARPin 53.2F11[TEV], which is the same DARPin as 53.2.F11 but harbors a TEV cleavage site between the His-tag and the HA-tag, to immobilized human CD32a protein with an affinity (KD) in the lower nanomolar range (<2×10⁻⁹ M), while the affinity for human CD32b was not evaluable (below baseline) (FIG. 10). For all other DARPin candidates (53.H3, 53.D8, 53.2.F11, 52.H6, 53.A8, 53.C8) tested, similar affinities for CD32a were observed (<2×10⁻⁹ M), while binding to CD32b was always below baseline.

1. A binding protein comprising at least one ankyrin repeat domain, wherein said at least one ankyrin repeat domain specifically binds CD32a, and wherein said at least one ankyrin repeat domain comprises at least one ankyrin repeat unit having an amino acid sequence selected from the group consisting of
i) SEQ ID: 28, 29 and 30, and
ii) SEQ ID NO: 28, 29 and 30 independent of each other comprising up to 10 amino acid substitutions, wherein preferably the substitutions are independent of each other at positions 1, 3, 4, 6, 14, 15, and/or 27 of the amino acid sequence according to SEQ ID NO: 28, 29 and 30 and/or independent of each other at up to three other positions the amino acid sequence according to SEQ ID NO: 28, 29 and 30.

2. A binding protein comprising at least one ankyrin repeat domain, wherein said at least one ankyrin repeat domain specifically binds CD32a, and
wherein said at least one ankyrin repeat domain comprises an amino acid sequence that has at least 70% amino acid sequence identity with one ankyrin repeat domain selected from the group consisting of the amino acid sequence according to SEQ ID NOs: 1-27.

3. The binding protein of claim 1, wherein said at least one ankyrin repeat domain comprises at least one ankyrin repeat unit consisting of an amino acid sequence according to SEQ ID NO: 31
(SEQ ID NO: 31)
X1DX2X3GX4TPLHLAAX5X6GHLEIVEVLLKX7GADVNA
wherein
X1 to X6 can be any amino acid except for glycine, proline or cysteine, and
X7 can be histidine, asparagine or tyrosine, and
wherein optionally up to 5 amino acids in other than in positions denoted with X in SEQ ID NO: 31 are substituted by any amino acid.

4. The binding protein of claim 3, wherein
X1 is T, E, N, 5, Y, or W,
X2 is E, N, T, W, H, K, N, or I,
X3 is E, F, K, H, M, A, or T,
X4 is L, T, A, V, F, Q, or A,
X5 is L, Q, V, E, M, V, or Y,
X6 is I, F, E, Q, H, V, W, or D, and
X7 is Y, N, or H.

5. The binding protein of claim 3, wherein
X1 is A, H, M, T, D, Q, S, or V,
X2 is M, Y, N, E, W, D, H, or T,
X3 is D, W, Q, S, V, F, E, or H,
X4 is T, R, Q, N, M, D, S, Y, or P,
X5 is Q, E, V, H, T, R, M, or Y,
X6 is E, Y, W, F, or A,
X7 is Y, or N, and
wherein the amino acids starting from and including position 15 are optionally missing.

6. The binding protein of any one of claim 3, wherein
X1 is D, A, S, Q, H, or Y,
X2 is F, I, H, V, Y, T, V, C,
X3 is W, A, F, V, E, or G,
X4 is H, Y, L, M, or F,
X5 is W, F, I, H, or R,
X6 is F, I, Q, D, or L, and
X7 is H, Y, or N.

7. The binding protein of claim 3, further comprising a N-terminal and/or C-terminal capping repeat domain, preferably wherein the N-terminal and/or C-terminal capping repeat domain have an amino acid sequence different from any one of the ankyrin repeat units.

8. The binding protein of claim 1, wherein said binding protein comprises an antigen binding protein, preferably it comprises a DARPin or is a DARPin.

9. A nucleic acid comprising a nucleic acid encoding the binding protein of claim 1.

10. A vector comprising the nucleic acid of claim 9.

11. A vector comprising the binding protein of claim 1.

12. A pharmaceutical composition comprising at least one binding protein of claim 1 and a pharmaceutically acceptable carrier.

13-14. (canceled)

15. A kit comprising the binding protein of claim 1.

16. The binding protein of claim 4, wherein
X1 is T,
X2 is E,
X3 is E,
X4 is L,
X5 is L,
X6 is I, and
X7 is Y.

17. The binding protein of claim 5, wherein
X1 is A,
X2 is M,
X3 is D,
X4 is T,
X5 is Q,
X6 is E,
X7 is Y, and
wherein the amino acids starting from and including position 15 are optionally missing.

18. The binding protein of any one of claim 6, wherein
X1 is D,
X2 is F,
X3 is W,
X4 is H,
X5 is W,
X6 is F, and
X7 is H.

19. A pharmaceutical composition comprising the nucleic acid of claim 9 and a pharmaceutically acceptable carrier.

20. A method for treating, preventing, or diagnosing a condition associated with altered CD32a levels in a subject in need thereof, comprising administering the binding protein of claim 1 to the subject.

21. The method of claim 20, wherein said condition associated with altered CD32a is a HIV related disorder.

22. A method for treating, preventing, or diagnosing a condition associated with altered CD32a levels in a subject in need thereof, comprising administering the nucleic acid of claim 9, to the subject.
